(19) United States
(12) Patent Application Publication
(10) Pub. No.: US 2012/0208762 A1
(43) Pub. Date: Aug. 16, 2012

(54) Methods of Diagnosing Diastolic Dysfunction

(75) Inventor: Samuel Dudley, Chicago, IL (US)

(73) Assignee: The Board of Trustees of the University of Illinois, Urbana, IL (US)

(21) Appl. No.: 13/503,812

(22) PCT Filed: Oct. 26, 2010
(86) PCT No.: PCT/US2010/054096

§ 371 Date: Apr. 24, 2012

Related U.S. Application Data
(60) Provisional application No. 61/255,381, filed on Oct. 27, 2009.
(60) Provisional application No. 61/359,695, filed on Jun. 29, 2010.

(51) Int. Cl. A61K 38/00; G01N 33/566; A61P 9/00; G01N 33/573; A61K 31/7088

(52) U.S. Cl. 514/16.4; 435/7.4; 514/44 R; 514/1.1; 436/501

(57) ABSTRACT

Diagnostic methods relating to a cardiac ventricular dysfunction are provided. In some embodiments, the diagnostic method is a method of diagnosing diastolic dysfunction in the absence of systolic dysfunction in a subject. The method comprises assaying a sample obtained from the subject for evidence of activation of renin-angiontensin system (RAS), evidence of oxidative stress, a level of adiponectin, or a combination thereof, wherein, when there is a lack of evidence of RAS activation, a lack of evidence of oxidative stress, a reduction in the level of adiponectin, or a combination thereof, as compared to a control subject, the subject is diagnosed with diastolic dysfunction in the absence of systolic dysfunction. A method of diagnosing a type of cardiac ventricular dysfunction, a method of determining a therapeutic regimen for a subject suffering from a cardiac ventricular dysfunction and methods of treating diastolic dysfunction in the absence of systolic dysfunction are also provided.

### CROSS-REFERENCE TO RELATED APPLICATIONS

This application claims priority to U.S. Provisional Patent Application No. 61/255,381, filed on Oct. 27, 2009, and U.S. Provisional Patent Application No. 61/359,695, filed on Jun. 29, 2010, each of which is incorporated by reference in their entirety.

### GRANT FUNDING

This invention was made with government support under Grant Nos. RO1 HL 085558, RO1 HL 085520, RO1 HL 073753, and P01 HL 058000, awarded by the National Institutes of Health. The government has certain rights in the invention.

### BACKGROUND

Patients presenting signs and/or symptoms of heart failure may be suffering from systolic dysfunction, diastolic dysfunction, or both types of dysfunction. Successful treatment of the patient depends on the type of cardiac dysfunction present, since the treatment of diastolic dysfunction without systolic dysfunction is very different from the treatments used for patients presenting with systolic dysfunction (with or without systolic dysfunction).

Methods of diagnosing diastolic dysfunction are known in the art and include, for example, Doppler echocardiography, cardiac catheterization, magnetic resonance imaging, tissue Doppler imaging, and measurement of left ventricular end diastolic pressure and systolic function. However, these methods can be invasive, time-consuming, and costly. Accordingly, there exists a need in the art for non-invasive, time-effective, and cost-effective methods of accurately diagnosing diastolic dysfunction in the absence of systolic dysfunction. Such methods would also facilitate practitioners to choose the appropriate treatment for the patient.

### SUMMARY

Presented herein for the first time are data which demonstrate that diastolic dysfunction in the absence of systolic dysfunction is associated with neither activation of the renin-angiontensisn system (RAS) nor oxidative stress. Also presented herein for the first time are data which demonstrate that diastolic dysfunction in the absence of systolic dysfunction is associated with reduced levels of adiponectin. Accordingly, provided herein are diagnostic methods relating to a cardiac dysfunction, e.g., diastolic dysfunction, systolic dysfunction. In some embodiments, the diagnostic method is a method of diagnosing diastolic dysfunction in the absence of systolic dysfunction in a subject, e.g., a subject exhibiting a sign or symptom of heart failure. The method comprises assaying a sample obtained from the subject for evidence of activation of renin-angiontensin system (RAS), evidence of oxidative stress, a level of adiponectin, or a combination thereof. In some embodiments, the subject is diagnosed with diastolic dysfunction in the absence of systolic dysfunction, when there is a lack of evidence of RAS activation, a lack of evidence of oxidative stress, a reduction in the level of adiponectin, or a combination thereof, as compared to a control subject, e.g., a control subject exhibiting a sign or symptom of heart failure.

In some embodiments, the diagnostic method of the present disclosures is a method of diagnosing a type of heart failure in a subject suffering from a heart failure. The method comprises assaying a sample obtained from the subject for evidence of activation of renin-angiontensin system (RAS), evidence of oxidative stress, a level of adiponectin, or a combination thereof. In some embodiments, the subject is diagnosed with heart failure with preserved ejection fraction, e.g., diastolic heart failure, when there is a lack of evidence of RAS activation, a lack of evidence of oxidative stress, a reduction in the level of adiponectin, or a combination thereof, as compared to a control subject, e.g., a control subject suffering from heart failure (e.g., systolic heart failure, heart failure with systolic dysfunction).

Further provided herein is a method of determining a therapeutic regimen for a subject exhibiting a sign or symptom of heart failure. The method comprises assaying a sample obtained from the subject for evidence of activation of renin-angiontensin system (RAS), evidence of oxidative stress, a level of adiponectin, or a combination thereof. In some embodiments, the therapeutic regimen is determined to be a therapeutic regimen for treating diastolic dysfunction in the absence of systolic dysfunction, when there is a lack of evidence of RAS activation, a lack of evidence of oxidative stress, a reduction in the level of adiponectin, or a combination thereof, as compared to a control subject, e.g., a control subject exhibiting a sign or symptom of heart failure.

A method of treating a subject for diastolic dysfunction in the absence of systolic dysfunction is furthermore provided herein. The method comprises (a) assaying a sample obtained from the subject for evidence of activation of renin-angiontensin system (RAS), evidence of oxidative stress, a level of adiponectin, or a combination thereof, and (b) administering to the subject a therapeutic agent suitable for treating diastolic dysfunction in the absence of systolic dysfunction in an amount effective to treat the diastolic dysfunction.

Moreover, provided herein is a method of treating diastolic dysfunction in the absence of systolic dysfunction in a subject, comprising administering to the subject an agent which increases the level of adiponectin in the subject.

Further provided herein is a method of treating or preventing heart failure with preserved ejection fraction in a subject. The method comprises administering to the subject an agent which increases the level of adiponectin in the subject.

### BRIEF DESCRIPTION OF THE DRAWINGS

FIG. 1 represents a graph of the multivariate odds ratios for association with early diastolic dysfunction {BMI, Body mass index; Eₕ CyS , Redox potential of reduced to oxidized cysteine; Eₕ GSH, Redox potential of reduced to oxidized glutathione; DROM , Derivatives of reactive oxygen metabolites; IsoP, Isoprostanes; ACE , Angiotensin converting enzyme levels.}

FIG. 2 represents a Western blot demonstrating the protein expression of ecSOD in blood samples from DD and control groups. The results depicted in the graph are presented as mean ±SE (DD (n=6) and controls (n=12)).

FIG. 3 represents a series of graphs demonstrating a comparison of (Top) total, (Center) high molecular weight, and (Bottom) mid+low molecular weight adiponectin levels between patients with and without diastolic dysfunction (DD).

FIG. 4 represents a series of graphs demonstrating a correlation of body mass index with (Top) total, (Center) high molecular weight, and (Bottom) mid+low molecular weight adiponectin levels. HMW, high molecular weight fraction; MMW+LMW, Mid and low molecular weight fractions; Dotted lines show 95% confidence intervals for individual cases.

### DETAILED DESCRIPTION

Provided herein are diagnostic methods relating to a cardiac dysfunction, e.g., diastolic dysfunction, systolic function. As used herein, the term “diagnose” and words stemming therefrom refer to a determination of the presence or absence of a medical condition, disease, or syndrome in a subject. In some embodiments, the diagnosis achieved through the methods of the present disclosures comprises a determination of the presence of a first medical condition, disease, or syndrome and a determination of the absence of one or more medical conditions, diseases, or syndromes. In some embodiments, the diagnostic methods provided herein comprises a determination of the stage, class, or type of the medical condition, disease, or syndrome in a subject. In some embodiments, the diagnostic methods provided herein comprises a determination of a susceptibility to the medical condition, a disease, or syndrome in the subject.

In some embodiments, the diagnostic method is a method of diagnosing diastolic dysfunction in the absence of systolic dysfunction in a subject, e.g., a subject exhibiting a sign or symptom of heart failure. The method comprises assaying a sample obtained from the subject for evidence of activation of renin-angiontensin system (RAS), evidence of oxidative stress, a level of adiponectin, or a combination thereof. In some embodiments, the subject is diagnosed with diastolic dysfunction in the absence of systolic dysfunction, when there is a lack of evidence of RAS activation, a lack of evidence of oxidative stress, a reduction in the level of adiponectin, or a combination thereof, as compared to a control subject, e.g., a control subject exhibiting a sign or symptom of heart failure.

Diastolic Dysfunction

As used herein, the term “diastolic dysfunction” refers to a condition in which abnormalities in mechanical function are present during diastole. Diastolic dysfunction can occur in the presence or absence of heart failure and can co-exist with or without abnormalities in systolic function (Zile et al., JACC 41: 1519-1522 (2003)). Accordingly, in some embodiments, the diastolic dysfunction which is diagnosed by the methods of the present disclosures is diastolic dysfunction in the absence of systolic dysfunction, which is also known as, diastolic dysfunction with preserved ejection fraction, diastolic dysfunction with preserved systolic function, and diastolic dysfunction with preserved left ventricular function. As used herein, the term “preserved ejection fraction” refers to a left ventricular ejection fraction which is greater than or about 45%, e.g., greater than or about 50%. In some aspects, the preserved ejection fraction is one which is greater than or about 50%. In some embodiments, the diastolic dysfunction is an early diastolic dysfunction. As used herein, the term “early diastolic dysfunction” refers to a medical condition in which ventricle filling is impaired as evidenced by the ratio of the peak velocities of blood across the mitral valve in diastole in early filling, the E wave to that during atrial contraction, the A wave, (E/A ratio)≦1 and peak early (E′) and late (A′) mitral annular velocities recorded by conventional pulsed wave Doppler method also≦1 (Vasan et al., J Am Coll Cardiol 26:1565-1574 (1995); Xie et al., J Am Coll Cardiol 24:132-139 (1994); Moller et al., J Am Coll Cardiol 35:363-370 (2000)). In some aspects, the diastolic dysfunction is characterized by (i) a lack of increased late I_{Na} in cardiomyocytes, (ii) an increase in myofilament calcium sensitivity, or (iii) a combination thereof.

Systolic Dysfunction

In some embodiments, the diagnostic method provided by the present disclosures relates to systolic dysfunction. In simple terms, systolic dysfunction is a condition in which the pump function or contraction of the heart (i.e., systole), fails. Systolic dysfunction may be characterized by a decreased or reduced ejection fraction, e.g., an ejection fraction which is less than 45%, and an increased ventricular end-diastolic pressure and volume. In some aspects, the strength of ventricular contraction is weakened and insufficient for creating an appropriate stroke volume, resulting in less cardiac output.

Accordingly, in some aspects, the diagnostic method is a method of ruling out systolic dysfunction in a subject or a method of diagnosing the absence of systolic dysfunction in a subject, e.g., a subject exhibiting a sign or symptom of heart failure. The method comprises assaying a sample obtained from the subject for evidence of activation of renin-angiontensin system (RAS), evidence of oxidative stress, a level of adiponectin, or a combination thereof. In some embodiments, the subject is diagnosed with the absence of systolic dysfunction, when there is a lack of evidence of RAS activation, a lack of evidence of oxidative stress, a reduction in the level of adiponectin, or a combination thereof, as compared to a control subject, e.g., a control subject exhibiting a sign or symptom of heart failure. In some aspects, the subject is further diagnosed as having diastolic dysfunction.

Heart Failure

Heart failure (HF) is defined as the ability of the heart to supply sufficient blood flow to meet the body's needs. In some embodiments, the signs and symptoms of heart failure include dyspnea (e.g., orthopnea, paroxysmal nocturnal dyspnea), coughing, cardiac asthma, wheezing, dizziness, confusion, cool extremities at rest, chronic venous congestion, ankle swelling, peripheral edema or anasarca, nocturia, ascites, heptomegaly, jaundice, coagulopathy, fatigue, exercise intolerance, jugular venous distension, pulmonary rales, peripheral edema, pulmonary vascular redistribution, interstitial edema, pleural effusions, or a combination thereof. In some embodiments, the signs and symptoms of heart failure include dyspnea (e.g., orthopnea, paroxysmal nocturnal dyspnea), fatigue, exercise intolerance, jugular venous distension, pulmonary rales, peripheral edema, pulmonary vascular redistribution, interstitial edema, pleural effusions, or a combination thereof. In some embodiments, the symptom of heart failure is one of the symptoms listed in the following table, which provides a basis for classification of heart failure according to the New York Heart Association (NYHA).

| NYHA | |
|---|---|
| Class | Symptoms |
| I | No symptoms and no limitation in ordinary physical activity, |
| | e.g. shortness of breath when walking, climbing stairs etc. |
| II | Mild symptoms (mild shortness of breath and/or angina) |
| | and slight limitation during ordinary activity. |
| III | Marked limitation in activity due to symptoms, even during |
| | less-than-ordinary activity, e.g. walking short distances |
| | (20-100 m). Comfortable only at rest. |
| IV | Severe limitations. Experiences symptoms even while at rest. |
| | Mostly bedbound patients. |

Patients presenting with signs and/or symptoms of heart failure may be suffering from systolic dysfunction, diastolic dysfunction, or a combination of the two. Because the diagnostic methods provided herein are related to diastolic dysfunction and systolic dysfunction, and, because, diastolic dysfunction and systolic dysfunction can lead to heart failure, the present disclosures also provide a method of diagnosing a type of heart failure in a subject suffering from heart failure. The method comprises assaying a sample obtained from the subject for evidence of activation of renin-angiontensin system (RAS), evidence of oxidative stress, a level of adiponectin, or a combination thereof. In some embodiments, the subject is diagnosed with heart failure with preserved ejection fraction, when there is a lack of evidence of RAS activation, a lack of evidence of oxidative stress, a reduction in the level of adiponectin, or a combination thereof, as compared to a control subject. Heart failure with preserved ejection fraction, which is also known as, heart failure with preserved systolic function, heart failure without systolic dysfunction, and heart failure with preserved left ventricular function, is a clinical condition in which the subject exhibits a preserved ejection fraction (e.g., an ejection fraction which is greater than or about 45%, or greater than or about 50%) along with signs and/or symptoms of heart failure.

In some embodiments, the heart failure is acute heart failure with preserved ejection fraction. In some embodiments, the heart failure is chronic heart failure with preserved ejection fraction. In some embodiments, the heart failure is acute and chronic heart failure with preserved ejection fraction.

In some embodiments, the heart failure which is diagnosed is a Class I, Class II, Class III, or Class IV heart failure as defined by the New York Heart Association (NYHA). See, for example, The Criteria Committee of the New York Heart Association. Nomenclature and Criteria for Diagnosis of Diseases of the Heart and Great Vessels. 9th ed. Boston, Mass: Little, Brown & Co; 1994:253-256, and the table above. In some embodiments, the heart failure is an NYHA Class I or Class II heart failure.

RAS Activation

In some embodiments, the diagnostic methods provided herein comprise assaying a sample obtained from the subject for evidence of activation of the renin-angiotensin system (RAS). As used herein, the term “renin-angiotensin system” or “RAS” is synonymous with “RAAS” or “renin-angiotensin-aldosterone system” and refers to the biological pathways that are activated in response to decreased blood volume. When blood volume is low, the kidneys produce renin, which stimulates the production of angiotensin from angiotensinogen. Angiotensin in turn causes vasoconstriction, resulting in increased blood pressure. Angiotensin also causes secretion of aldosterone from the adrenal cortex. Aldosterone causes the tubules of the kidneys to increase reabsorption of sodium and water into the blood, which increases the volume of fluid in the body, thereby increasing blood pressure.

In some embodiments, assaying the sample for evidence of RAS activation comprises assaying for one or more positive RAS markers, one or more negative RAS markers, or a combination thereof. As used herein, the term “positive RAS marker” refers to a marker of which the level or activity increases in response to RAS activation. With regard to the methods of the present disclosures, a lack of an increase in one or more positive RAS markers is indicative of a lack of evidence of RAS activation, which, according to the present disclosures, is indicative of the subject suffering from diastolic dysfunction in the absence of systolic dysfunction. In exemplary embodiments, the positive RAS marker is renin, angiotensin II, aldosterone, angiotensin converting enzyme (ACE), NADPH oxidase, or a combination thereof. In exemplary aspects, the method of diagnosing diastolic dysfunction in the absence of systolic dysfunction comprises assaying the sample for one or more of these positive RAS markers. In specific aspects, the method comprises assaying for concentrations or amounts of ACE (e.g., ACE protein, ACE activity, ACE mRNA).

As used herein, the term “negative RAS marker” refers to a marker of which the level or activity decreases in response to RAS activation. With regard to the methods of the present disclosures, a lack of a decrease in one or more negative RAS markers is indicative of a lack of evidence of RAS activation, which, according to the present disclosures, is indicative of the subject suffering from diastolic dysfunction in the absence of systolic dysfunction. In exemplary aspects, the method of diagnosing diastolic dysfunction in the absence of systolic dysfunction comprises assaying the sample for one or more of these negative RAS markers.

In some embodiments, the method comprises assaying for one or more positive RAS markers, one or more negative RAS markers, or a combination thereof. In some aspects, the method comprises assaying for only positive RAS markers. In alternative aspects, the method comprises assaying for only negative RAS markers. In yet alternative aspects, the method comprises assaying for both positive RAS markers and negative RAS markers. In exemplary embodiments, the method comprises assaying for angiotensin I, angiotensinogen, anti-diuretic hormone (ADH), which is also known as vasopres sin, leptin, resistin, or a combination thereof, optionally, in combination with any of the positive or negative RAS markers described above.

Oxidative Stress

Oxidative stress represents a condition of a biological system in which the production of reactive oxygen species (ROS) outweighs the biological system's ability to detoxify the ROS or reactive intermediates thereof or to repair the damage induced by the ROS or reactive intermediates thereof. Oxidative stress may be characterized as an increase in production of oxidative species, e.g., ROS, or a decrease in the capability of an antioxidant defense and may lead to cellular death, e.g., apoptosis, necrosis.

Oxidative stress is related to RAS insofar as Angiotensin II of RAS activates NADPH oxidase to produce reactive oxygen species (ROS), which in turn oxidizes NO, which causes vasodilation. When RAS is too active, blood pressure is too high and an overproduction of ROS may occur. The over-activation of RAS may result in hypertension and oxidative stress.

In some embodiments of the present disclosures, assaying the sample for evidence of oxidative stress comprises assaying for one or more positive oxidative stress markers, one or more negative oxidative stress markers, or a combination thereof. As used herein, the term “positive oxidative stress marker” refers to a marker of which the level or activity increases in response to oxidative stress. With regard to the methods of the present disclosures, a lack of an increase in one or more positive oxidative stress markers is indicative of a lack of evidence of oxidative stress activation, which, according to the present disclosures, is indicative of the subject suffering from diastolic dysfunction in the absence of systolic dysfunction. In exemplary embodiments, the positive oxidative stress marker is a ROS, glutathione disulfide (GSSG), oxidized cystine (CysS), a lipid peroxidase, an isoprostane, nitrite, nitrate, plasminogen activator inhibitor (PAI-1), dihydrobiopterin (BH₂), uncoupled nitric oxide synthase, or a combination thereof. In some embodiments, uncoupled nitric oxide synthase refers to a monomeric or unbound form of nitric oxide synthase.

In some embodiments, the ROS is a peroxide, e.g., any compound containing the peroxide anion (O₂²⁻) or an oxygen-oxygen single bond. In some embodiments, the ROS is a free radical, e.g., an atom, molecule, or ion with unpaired elections on an open shell configuration. In some aspects, the ROS is a superoxide anion, hydrogen peroxide, hydroxyl radical, organic hydroperoxide, alkyoxy radical, peroxy radical, hypochlorous acid, peroxynitrite.

In some embodiments, the oxidative stress marker is a product compound or product molecule resulting from a ROS reacting with an organic substrate (e.g., carbohydrate, lipid, amino acid, protein, peptide, nucleotide, nucleic acid, etc.). In exemplary aspects, the oxidative stress marker is a lipid peroxide, e.g., a peroxidized arachidonic acid.

In some embodiments, the isoprostane is a prostaglandin-like compound formed in vivo via a non-enzymatic mechanism involving the free radical-initiated peroxidation of arachidonic acid. In some aspects, the isoprostane is an F2 isoprostane, is a furan or dioxolane ring isoprostane generated from arachidonic acid. In some aspects, the F2 isoprostane is a compound of the 5-series of F2 isoprostanes, a compound of the 12-series of F2 isoprostanes, a compound of the 8-series of F2 isoprostanes, or a compound of the 15-series of F2 isoprostanes. In some aspects, the F2 isoprostane is 8-iso-prostaglandin F2 alpha.

In exemplary aspects, the method of diagnosing diastolic dysfunction in the absence of systolic dysfunction comprises assaying the sample for one or more of these positive oxidative stress markers. In specific aspects, the method comprises assaying for concentrations or amounts of one or more of a lipid peroxide, an isoprotane (e.g., an F2 isoprostane), GSSG, and CysS.

As used herein, the term “negative oxidative stress marker” refers to a marker of which the level or activity decreases in response to oxidative stress activation. With regard to the methods of the present disclosures, a lack of a decrease in one or more negative oxidative stress markers is indicative of a lack of evidence of oxidative stress activation, which, according to the present disclosures, is indicative of the subject suffering from diastolic dysfunction in the absence of systolic dysfunction. In exemplary embodiments, the negative oxidative stress marker is glutathione (GSH), cysteine (reduced cysteine; Cys), nitric oxide (NO), a coupled nitric oxide synthase (NOS), tetrahydrobiopterin (BH₄), or a combination thereof. In exemplary aspects, the method of diagnosing diastolic dysfunction in the absence of systolic dysfunction comprises assaying the sample for one or more of these negative oxidative stress markers. In specific aspects, the method comprises assaying for concentrations or amounts of GSH and Cys. In some embodiments, a coupled NOS refers to a dimeric or multimeric or bound form of NOS.

In some embodiments, the method comprises assaying for one or more positive oxidative stress markers, one or more negative oxidative stress markers, or a combination thereof. In some aspects, the method comprises assaying for only positive oxidative stress markers. In alternative aspects, the method comprises assaying for only negative oxidative stress markers. In yet alternative aspects, the method comprises assaying for both positive oxidative stress markers and negative oxidative stress markers. In exemplary aspects, the method comprises assaying for GSH, GSSG, Cys, CysS, a lipid peroxidase, an F2 isoprostane, or a combination thereof. In exemplary embodiments, the method comprises assaying for angiotensin I, angiotensinogen, anti-diuretic hormone (ADH), which is also known as vasopressin, leptin, resistin, or a combination thereof, optionally, in combination with any of the positive oxidative stress markers, negative oxidative stress markers, positive RAS markers, and/or negative RAS markers described above.

Adiponectin

Adiponectin is a multimeric adipokine exclusively expressed in adipose tissue. Adiponectin is one of the most abundant plasma proteins in humans. The human amino acid sequence of this protein is publicly available in the Protein database of the National Center for Biotechnology Information (NCBI) website as Accession No. NP_{—}001171271 and is provided herein as SEQ ID NO: 1.

In some embodiments of the present disclosures, the method comprises assaying the sample for a level of adiponectin. In some aspects, assaying for a level of adiponectin comprises assaying the sample for a total level of adiponectin, a level of high molecular weight (HMW) adiponectin, a level of mid molecular weight (MMW) adiponectin, a level of low molecular weight (LMW) adiponectin, or a combination thereof. In some aspects, the method comprises assaying for a total level of adiponectin and a level of high molecular weight (HMW) adiponectin. In certain aspects, the method comprises calculating the sum of the levels of MMW adiponectin and LMW adiponectin by subtracting the level of high molecular weight (HMW) adiponectin (e.g., as measured in the sample) from the total level of adiponectin (e.g., as measured in the sample).

In some embodiments, the total level of adiponectin is the total level of adiponectin found in plasma. In some embodiments, the level of HMW adiponectin is the level of HMW adiponectin found in plasma. In some aspects, the total level of adiponectin and the HMW adiponectin level are assayed in accordance with the steps described in EXAMPLE 3.

Measurement of Markers

With regard to the present disclosures, the term “marker” refers to any chemical or biological compound including, but not limited to amino acids, peptides, proteins, nucleotides, nucleic acids, DNA, RNA, lipids, carbohydrates, sugars, organic small molecules. In some aspects, the measurement of a marker, e.g., a positive RAS marker, a negative RAS marker, a positive oxidative stress marker, a negative oxidative stress marker, adiponectin, comprises assaying or determining the level, concentration, or amount of the marker. In some embodiments, the level, concentration or amount of the marker is an absolute level, concentration, or amount. In certain aspects, the absolute level, concentration, or amount is a quantification of the protein level. In alternative embodiments, the level, concentration or amount of the marker is a relative level, relative concentration, or relative amount. In some aspects, the relative level is expressed as a ratio. In some embodiments, the level, concentration, or amount of the marker is represented by the activity of a biological molecule related to the marker, as further described herein.

In some aspects, the marker is a protein and measurement of the protein in some embodiments comprises assaying for or determining the protein level in the sample. In some aspects, the protein level is determined by an immunoassay, e.g., Western blotting, an enzyme-linked immunosorbent assay (ELISA), a radioimmunoassay (RIA), an immunohistochemical assay, which methods are known in the art.

In some aspects, the protein level is represented by the expression level of the gene encoding the protein. In some aspects, the marker is a gene or nucleic acid molecule encoding a protein. In such aspects, measurement of the marker comprises assaying for levels of the nucleic acid, e.g., mRNA, in the sample.

In some aspects, the protein level is represented by a level of the protein's biological activity, e.g., enzymatic activity. In exemplary aspects, the protein level is reflected by the levels of the substrate or product of the enzymatic reaction catalyzed by the protein marker. Methods of assaying for the level of biological activity, e.g., enzymatic activity, are known in the art, and include, the ACE activity assay described in the EXAMPLES section set forth below.

In some aspects, the protein level is represented by the level of biological activity of a related protein, e.g., a protein which acts upstream or downstream of the marker. For example, if the marker is a phosphorylated protein in the active state, then, in some embodiments, the marker level is represented by the activity level of the kinase which phosphorylates the marker. In other aspects, if the marker is a transcription factor which activates expression of a gene, then, in some embodiments, the marker level is represented by the expression levels of the gene activated by the marker.

When the marker is neither a protein nor a nucleic acid, and is, for example, a lipid, carbohydrate, sugar, organic small molecule, measurement of the marker in some embodiments comprises measuring the level, amount, or concentration of marker through methods known in the art, e.g., chromatography, mass spectrometry. The chromatography in some aspects is any of a column chromatography, a planar chromatography (e.g., paper chromatography, thin layer chromatography), gas chromatography, displacement chromatography, liquid chromatography (e.g., high performance liquid chromatography (HPLC)), affinity chromatography, supercritical fluid chromatography, ion exchange chromatography, size exclusion chromatography, reversed phase chromatography, two dimensional chromatography, simulated moving bed chromatography, pyrolysis gas chromatography, and the like. In exemplary embodiments, when the marker is a DROM, a d-ROMs test (Diacron International, Grosseto, Italy) may be used to measure the level of the DROM in accordance with the manufacturer's instructions. If the marker is an isoprostane, e.g., an F2 isoprostane, the marker level may be determined through the methods described in the art, e.g., gas chromatography/mass spectrometry/negative ion chemical ionization. See, for example, Nourooz-Zadeh, Biochem Society Transactions 36: 1060-1065 (2008) and EXAMPLES section set forth below.

In alternative embodiments in which the marker is neither a protein nor a nucleic acid, the measurement of the marker comprises measurement of the proteins which catalyze the production of the marker, or the catalytic activity of such proteins.

Additional Steps

In some embodiments of the diagnostic methods provided herein, the method comprises additional steps or comprises a combination of the steps disclosed herein. In exemplary embodiments, the diagnostic methods comprise assaying for all of evidence of RAS activation, evidence of oxidative stress, and a level of adiponectin. As used herein, the term “combination” encompasses all of the listed individual elements or a sub-combination thereof. In some aspects, the diagnostic methods comprise a combination of assaying the sample for a positive RAS marker, a negative RAS marker, a positive oxidative stress marker, a negative oxidative stress marker, and a level of adiponectin. Each combination and sub-combination are contemplated herein. In some aspects, the diagnostic methods comprise assaying for only evidence of RAS activation and evidence of oxidative stress. For example, in some aspects, the methods comprise assaying for only positive markers, e.g., positive RAS markers and positive oxidative stress markers. In some aspects, the methods comprise only assaying for negative markers, e.g., negative RAS markers and negative oxidative stress markers. In some aspects, the diagnostic methods comprise assaying for only a level of adiponectin, or only a level or adiponectin in combination with one of evidence of RAS activation or evidence of oxidative stress.

In some aspects, the method comprises one or more additional diagnostic steps. In some embodiments, the method comprises measuring left ventricle (LV) pressure, volume, and/or wall thickness. In some embodiments, the method comprises executing calculations that reflect the process of active relaxation (the rate of isovolumic LV pressure and LV filling) and calculations that reflect passive stiffness (chamber compliance and myocardial viscoelastic stiffness) (Zile et al., (2010), supra).

In some embodiments, the methods comprise performing an echocardiography, as described in Silberman et al., Circulation 121: 519-528 (2010). In some aspects, LV tissue Doppler and mitral valve in-flow velocity are measured by echocardiography. In some aspects, the method comprises assaying for a late diastolic velocity (A′) which is higher than the early diastolic velocity (E′), by tissue Doppler imaging (TDI).

In alternative or additional embodiments, the method comprises performing a magnetic resonance imaging (MRI), cardiac catheterization, or measurement of LV end diastolic pressure and systolic function.

In some embodiments, the method comprises additional steps which further characterize the subject. In exemplary aspects, the method comprises determining a body mass index (BMI) of the subject, performing a physical examination, performing a chest X-ray, or a combination thereof.

In some embodiments, the diagnostic methods of the present disclosures comprises a treatment step based on the outcome of the diagnosis. Accordingly, the present disclosures further provides a method of treating a subject for diastolic dysfunction in the absence of systolic dysfunction. The method comprises (a) assaying a sample obtained from the subject for evidence of activation of renin-angiontensin system (RAS), evidence of oxidative stress, a level of adiponectin, or a combination thereof, and (b) administering to the subject a therapeutic agent suitable for treating diastolic dysfunction in the absence of systolic dysfunction in an amount effective to treat the diastolic dysfunction, e.g., when (a) indicates a lack of evidence of RAS activation, a lack of evidence of oxidative stress, a reduction in the level of adiponectin, or a combination thereof, as compared to a control subject.

Therapeutic Agents for Treating Diastolic Dysfunction in the Absence of Systolic Dysfunction

The therapeutic agent suitable for treating the diastolic dysfunction in the absence of systolic dysfunction may be any medical standard of care for diastolic dysfunction in the absence of systolic dysfunction. In some aspects, the therapeutic agent is tetrahydrobiopterin, or a derivative thereof, such as any of those disclosed in U.S. Patent Application Publication No. 2008-0075666A1. In some aspects, the therapeutic agent is an agent which increases the level of adiponectin in the subject, such as any of those described herein. In some aspects, the therapeutic agent is a cardiac metabolic modifier in accordance with co-pending International Patent Application No. PCT/US2010/048650. Accordingly, the therapeutic agent in some embodiments comprises a structure of Formula I:

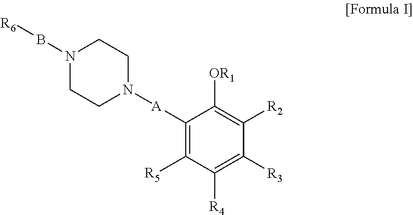

wherein A comprises a main chain of 1-8 atoms, each atom of which is independently C, O, N, or S, and each atom or which is optionally bound to an additional group selected from C1-C8 alkyl, C1-C8 alkoxy, OH, NH₂, NH(C1-C4 alkyl) and SH;

wherein R₁ is H or a C1-C8 alkyl;

wherein each of R₂, R₃, R₄, and R₅ independently is H, a C1-C8 alkyl, or a C1-C8 alkoxy;

wherein B is H or comprises a main chain of 1-8 atoms, each atom of which is independently C, O, N, or S, and each atom of which is optionally bound to an additional group; and,

wherein R₆ is absent or phenyl, which phenyl is optionally substituted with 1 to 5 groups, each group of which is independently C1-C8 alkyl, C1-C8 alkoxy, or OH.

As used herein, “alkyl” refers to straight chained and branched saturated hydrocarbon groups, nonlimiting examples of which include methyl, ethyl, and straight and branched propyl, butyl, pentyl, hexyl, heptyl, and octyl groups containing the indicated number of carbon atoms. The term Cn means the alkyl group has “n” carbon atoms. For example, C1-C7 alkyl refers to alkyl groups having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms).. Accordingly, the C1-C8 alkyl can be a methyl, ethyl, propyl, butyl, C5 alkyl, C6 alkyl, C7 alkyl, or C8 alkyl, of which the propyl, butyl, C5 alkyl, C6 alkyl, C7 alkyl, or C8 alkyl is a straight chain alkyl or branched alkyl.

As used herein “alkoxy” refers to —OR, wherein R is alkyl (e.g., a straight or branched chain alkyl group). Examples of alkoxy groups include, but are not limited to, methoxy, ethoxy, propoxy, isopropoxy, butoxy, n-butoxy, sec-butoxy, t-butoxy and the like. Accordingly, the C1-C8 alkoxy can be methoxy, ethoxy, C3 alkoxy, C4 alkoxy, C5 alkoxy, C6 alkoxy, C7 alkoxy, or C8 alkoxy, or which the C3 alkoxy, C4 alkoxy, C5 alkoxy, C6 alkoxy, C7 alkoxy, or C8 alkoxy is a straight chain alkoxy or branched alkoxy.

As used herein “NH(C1-C4 alkyl)” refers to nitrogen bound to both H and a C1-C4 alkyl.

With regard to Formula I, A comprises a main chain of 1-8 atoms, each atom of which is independently C, O, N, or S. In some aspects, A comprises a main chain of a single atom selected from C, O, N, and S. In some aspects, A comprises a main chain of 2-8 atoms (e.g., 2, 3, 4, 5, 6, 7, or 8 atoms), each atom of which is independently C, O, N, or S.

Each atom of the main chain of A is optionally bound to an additional group. In some aspects, the additional group is selected from C1-C8 alkyl, C1-C8 alkoxy, OH, NH2, NH(C1-C4 alkyl) and SH. In some aspects, every atom of the main chain is bound to an additional group. In other aspects, one or more, but not all, atoms of the main chain are bound to an additional group. In some instances, one atom of the main chain is bound to an additional group. In some instances, 2, 3, 4, 5, 6, or 7 atoms of the main chain is bound to an additional group.

In some aspects, A is (CH₂)₁₋₈. In some aspects, A is (CH₂)₁₋₆. In some aspects, A is (CH₂)₁₋₄. In some aspects, A is (CH₂)₁ or (CH₂)₂. In some aspects, A is (CH₂)₁.

In other aspects, A comprises a structure of Formula IV:

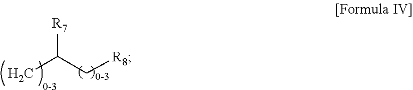

wherein R₇ is OH, C1-C4 alkyl, C1-C4 alkoxy, NH2, or NH(C1-C4 alkyl); and wherein R₈ is O or NH. In some aspects, when A comprises a structure of Formula IV, R₇ is OH and R₈ is O or NH. In some aspects, when A comprises a structure of Formula IV, R₇ is OH, C1-C4 alkyl, C1-C4 alkoxy, NH2, or NH(C1-C4 alkyl) and R₈ is O. In particular aspects, when A comprises a structure of Formula IV, R₇ is OH and R₈ is O. In some aspects, A comprises

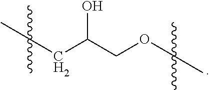

With regard to Formula I, R₁ is H or a C1-C8 alkyl. In particular aspects, R₁ is CH₃.

With regard to Formula I, each of R₂, R₃, R₄, and R₅ independently is H, a C1-C8 alkyl, or a C1-C8 alkoxy. In some aspects, each of R₂, R₃, R₄, and R₅ independently is H or methoxy. In alternative embodiments, each of R₂ and R₃ is a methoxy, and each of R₄ and R₅ is H.

With regard to Formula I, B is H or comprises a main chain of 1-8 atoms, each atom of which is independently C, O, N, or S. In some aspects, B comprises a main chain of a single atom selected from C, O, N, and S, while in other aspects, B comprises a main chain of 2-8 atoms (e.g., 2, 3, 4, 5, 6, 7, or 8 atoms), each atom of which is independently selected from C, O, N, and S.

Each atom of the main chain of B is optionally bound to an additional group. In some aspects, the additional group is selected from C1-C8 alkyl, C1-C8 alkoxy, OH, NH2, NH(C1-C4 alkyl) and SH. In some aspects, every atom of the main chain is bound to an additional group. In other aspects, one or more, but not all, atoms of the main chain are bound to an additional group. In some instances, one atom of the main chain is bound to an additional group. In some instances, 2, 3, 4, 5, 6, or 7 atoms of the main chain is bound to an additional group.

In some embodiments, B comprises H and R₆ is absent. In alternative embodiments, B comprises a structure of Formula V:

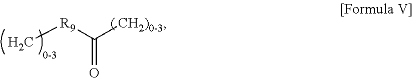

wherein R₉ is NH or O.

In some embodiments, when B comprises a structure of Formula V, R9 is NH. In further aspects, B comprises a structure of

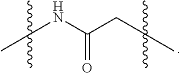

With regard to Formula I, R₆ is absent or phenyl, which phenyl is optionally substituted with 1 to 5 (e.g., 1, 2, 3, 4, 5) groups, each group of which is independently selected from C1-C8 alkyl, C1-C8 alkoxy, and OH. In some aspects, R₆ is absent. In alternative aspects, when R₆ is present and comprises phenyl substituted with 1 to 5 (e.g., 1, 2, 3, 4, 5) methyl groups. In specific aspects, R₆ comprises phenyl substituted with two methyl groups, one at each of the ortho positions.

In some aspects, the cardiac metabolic modifier comprises a compound of Formula II, or a pharmaceutically acceptable salt thereof or a conjugate thereof:

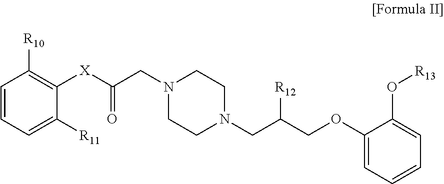

wherein each of R₁₀, R₁₁, and R₁₃ independently is C1-C3 alkyl, wherein X is NH or O; and wherein R₁₂ is OH or C1-C3 alkyl.

In some embodiments, the compound of Formula II comprises the following structure:

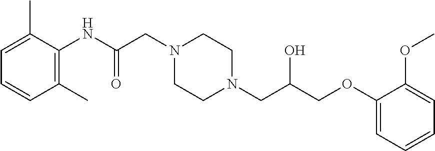

In some aspects, the compound of Formula II is ranolazine, or a pharmaceutically acceptable salt or a conjugate of ranolazine.

In some aspects, the cardiac metabolic modifier is a compound of Formula III, or a pharmaceutically acceptable salt thereof or a conjugate thereof:

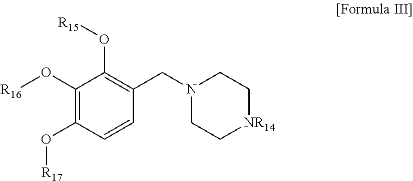

wherein each of R₁₄, R₁₅, R₁₆, and R₁₇ independently is H or C1-C3 alkyl.

In some embodiments, the compound of Formula II comprises the following structure:

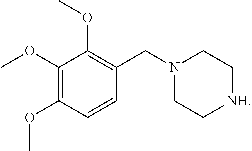

In some aspects, the compound of Formula III is trimetazidine, or a pharmaceutically acceptable salt or a conjugate of trimetazidine.

Therapeutic Regimen Determination

Since accurate diagnosis of a subject leads to determining the appropriate therapeutic regimen for treating the diagnosed medical condition, disease, or syndrome, the present disclosures accordingly provides a method of determining a therapeutic regimen for a subject suffering from diastolic from a cardiac ventricular dysfunction. The method comprises assaying a sample obtained from the subject for evidence of activation of renin-angiontensin system (RAS), evidence of oxidative stress, a level of adiponectin, or a combination thereof. In some embodiments, when there is a lack of evidence of RAS activation, a lack of evidence of oxidative stress, a reduction in the level of adiponectin, or a combination thereof, as compared to a control subject, the therapeutic regimen is determined to be a therapeutic regimen suitable for treating diastolic dysfunction in the absence of systolic dysfunction. In some embodiments, the therapeutic regimen comprises administration of a therapeutic agent suitable for treating diastolic dysfunction in the absence of systolic dysfunction in an amount effective to treat the diastolic dysfunction. Such therapeutic agents are known in the art and are disclosed herein. See, for example, the section set forth herein as “Therapeutic agents for treating diastolic dysfunction in the absence of systolic dysfunction.”

Agents which Increase Adiponectin Levels and Methods of Using Same

In addition to the diagnostic methods and related methods thereto described above, the present disclosures further provides a method of treating diastolic dysfunction in the absence of systolic dysfunction in a subject. The method comprises administering to the subject an agent which increases the level of adiponectin in the subject.

Because diastolic dysfunction can lead to heart failure with preserved ejection fraction, the invention also provides a method of treating or preventing heart failure with preserved ejection fraction. The method comprises administering to the subject an agent which increases the level of adiponectin in the subject.

In some embodiments, the agent which increases adiponectin levels is an adiponectin protein, a functional equivalent thereof, a nucleic acid molecule encoding adiponectin, or a functional equivalent thereof. The adiponectin protein in some aspects is a recombinant adiponectin protein. In some aspects, the adiponectin protein comprises the amino acid sequence of wildtype human adiponectin (SEQ ID NO: 1).

In some aspects, the agent is a functional equivalent of the adiponectin protein comprising an amino acid sequence which is at least or about 75% (e.g., at least or about 80%, at least or about 85%, at least or about 90%, at least or about 95%, at least or about 98%, at least or about 99%) identical to SEQ ID NO: 1 and substantially retains the biological activity of adiponectin, if not exceeds that of adiponectin, e.g., treats diastolic dysfunction in the absence of systolic function to a similar extent, the same extent, or to a higher extent, as adiponectin. The amino acid sequence of the functional equivalent in some embodiments comprises, for example, the amino acid sequence of SEQ ID NO: 1 with at least one conservative amino acid substitution. Conservative amino acid substitutions are known in the art, and include amino acid substitutions in which one amino acid having certain physical and/or chemical properties is exchanged for another amino acid that has the same chemical or physical properties. For instance, the conservative ammo acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional equivalent in some embodiments comprises the amino acid sequence of SEQ ID NO: 1 with at least one non-conservative amino acid substitution. In some aspects, the non-conservative amino acid substitution does not interfere with or inhibit the biological activity of the functional equivalent. In some aspects, the non-conservative amino acid substitution enhances the biological activity of the functional equivalent, such that the biological activity of the functional equivalent is increased as compared to adiponectin.

In some aspects, the functional equivalent is a functional fragment SEQ ID NO: 1 comprising at least or about 10 (e.g., at least or about 20, at least or about 30, at least or about 40, at least or about 50, at least or about 60, at least or about 70, at least or about80, at least or about 90, at least or about 100, at least or about 110, at least or about 120, at least or about 130, at least or about 140, at least or about 150, at least or about 160, at least or about 170, at least or about 180, at least or about 190, at least or about 200, at least or about 210, at least or about 220) contiguous amino acids of SEQ ID NO: 1 and treats diastolic dysfunction in the absence of systolic function to a similar extent, the same extent, or to a higher extent, as adiponectin.

The functional equivalent in some aspects comprises one or more synthetic amino acids in place of one or more naturally-occurring amino acids. Such synthetic amino acids are known in the art, and include, for example, aminocyclohexane carboxylic acid, norleucine, a-amino n- decanoic acid, homoserine, S-acetylammomethyl-cysteine, trans-3- and trans-4- hydroxyproline, 4-aminophenylalanine, 4- nitrophenylalanine, 4-chlorophenylalanine, 4- carboxyphenylalanine, 13-phenylserine13-hydroxyphenylalanine, phenylglycine, α- naphthylalanine, cyclohexylalanine, cyclohexylglycine, indoline-2-carboxylic acid, 1,2,3,4-tetrahydroisoquinoline-3 -carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, a-aminocyclohexane carboxylic acid, a- aminocycloheptane carboxylic acid, a-(2-amino-2-norbornane)-carboxylic acid, Δ,γdiaminobutyric acid, α,β-diaminopropionic acid, homophenylalanine, and α-tert-butylglycine.

In some embodiments, the adiponectin protein or functional equivalent thereof is glycosylated, amidated, carboxylated, phosphorylated, esterified, acetylated, N-acylated, cyclized via, e.g., a disulfide bridge, or converted into an acid addition salt and/or optionally dimerized or polymerized, or conjugated.

The adiponectin protein (including functional equivalents thereof) can be obtained by methods known in the art. Suitable methods of de novo synthesizing polypeptides and proteins are described in, for example, Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; and U.S. Patent No. 5,449,752.

Also, the adiponectin protein can be recombinantly produced using the nucleic acids described herein using standard recombinant methods. See, for instance, Sambrook et al., Molecular Cloning: A Laboratory Manual. 3^{rd} ed., Cold Spring Harbor Press, Cold Spring Harbor, N.Y., 2001; and Ausubel et al., Current Protocols in Molecular Biology, Greene Publishing Associates and John Wiley & Sons, NY, 1994.

Further, some of the adiponectin proteins (including functional equivalents thereof) can be isolated and/or purified, in part, from a source, such as a plant, a bacterium, an insect, a mammal, e.g., a rat, a human, etc. Methods of isolation and purification are well-known in the art.

Alternatively, the adiponectin proteins (including functional equivalents thereof) can be commercially synthesized by companies, such as Synpep (Dublin, Calif.), Peptide Technologies Corp. (Gaithersburg, Md.), and Multiple Peptide Systems (San Diego, Calif.). In this respect, the adiponectin proteins and functional equivalents thereof can be synthetic, recombinant, isolated, and/or purified.

In some embodiments, the agent which increases adiponectin levels is a nucleic acid molecule encoding wildtype human adiponectin (SEQ ID NO: 1) or a functional equivalent thereof. The term “nucleic acid molecule” as used herein is synonymous with “polynucleotide,” “oligonucleotide,” and “nucleic acid,” and generally means a polymer of DNA or RNA, which can be single-stranded or double- stranded, synthesized or obtained (e.g., isolated and/or purified) from natural sources, which can contain natural, non-natural or altered nucleotides, and which can contain a natural, non-natural or altered inter-nucleotide linkage, such as a phosphoroamidate linkage or a phosphorothioate linkage, instead of the phosphodiester found between the nucleotides of an unmodified oligonucleotide. In some aspects, the nucleic acid does not comprise any insertions, deletions, inversions, and/or substitutions. In other aspects, the nucleic acid comprises one or more insertions, deletions, inversions, and/or substitutions.

In some embodiments, the adiponectin-encoding nucleic acids are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication. The nucleic acids in some aspects are constructed based on chemical synthesis and/or enzymatic ligation reactions using procedures known in the art. See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5- carboxymethylaminomethyl-2-thiouridme, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N -substituted adenine, 7-methylguanine, 5-methylammomethyluracil, 5- methoxyaminomethyl-2-thiouracil, beta-D-mannosylqueosine, 5′- methoxycarboxymethyluracil, 5-methoxyuracil, 2-methylthio-N⁶-isopentenyladenine, uracil- 5-oxyacetic acid (v), wybutoxosine, pseudouratil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

In some aspects, the nucleic acid molecule is administered in the form of recombinant expression vector comprising the nucleic acid molecule. In alternative aspects, the nucleic acid molecule is administered by way of administering one or more host cells, each of which express the nucleic acid molecule.

Isolated and Purified

The adiponectin proteins and nucleic acid molecules, (including functional equivalents thereof) in some embodiments are isolated and/or purified. The term “isolated” as used herein means having been removed from its natural environment. The term “purified” as used herein means having been increased in purity, wherein “purity” is a relative term, and not to be necessarily construed as absolute purity. For example, the purity can be at least about 50%, can be greater than 60%, 70% or 80%, or can be 100%.

Treatment and Prevention

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of diastolic dysfunction or heart failure in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Samples

With regard to the diagnostic methods disclosed herein, in some embodiments, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of the foregoing samples. In some aspects, the sample is a composite panel of at least two of a blood sample, a plasma sample, a serum sample, and a urine sample.

Subjects

In some embodiments of the present disclosures, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In specific aspects, the human is a male human. In additional or alternative aspects, the human has a BMI of about 29 or higher.

In some embodiments, the subject exhibits or is presenting a sign or symptom of diastolic dysfunction. In exemplary embodiments, the subject exhibits an ejection fraction which is greater than or about 45%, e.g., greater than or about 50%, e.g., 50-75%.

In some embodiments, the subject exhibits or is presenting a sign or symptom of heart failure. In some aspects, the sign or symptom of heart failure is one of those previously described herein. See the section entitled Heart Failure.

In some embodiments, the subject is suffering from heart failure, e.g., diastolic heart failure, any of the types of heart failure described herein. See the section entitled Heart Failure.

In some embodiments, the subject suffers from hypertension. Hypertension is a chronic medical condition in which the systemic arterial blood pressure is elevated. The hypertension in some embodiments is classified as a primary hypertension for which no medical cause is found. In some embodiments, the hypertension is a secondary hypertension caused by another condition that affects the kidneys, arteries, heart, or endocrine system.

A systolic or the diastolic blood pressure measurement higher than the accepted normal values for the age of the individual is classified as prehypertension or hypertension.

| | Systolic pressure | | Diastolic pressure | |
|---|---|---|---|---|
| Classification | mmHg | kPa | mmHg | kPa |
| Normal | 90-119 | 12-15.9 | 60-79 | 8.0-10.5 |
| Prehypertension | 120-139 | 16.0-18.5 | 80-89 | 10.7-11.9 |
| Stage 1 | 140-159 | 18.7-21.2 | 90-99 | 12.0-13.2 |
| Stage 2 | ≧160 | ≧21.3 | ≧100 | ≧13.3 |
| Isolated systolic | ≧140 | ≧18.7 | <90 | <12.0 |
| hypertension | | | | |

Source: Chobanian et al. (2003)

Hypertension has several sub-classifications including, hypertension stage I, hypertension stage II, and isolated systolic hypertension. Isolated systolic hypertension refers to elevated systolic pressure with normal diastolic pressure and is common in the elderly. These classifications are made after averaging a patient's resting blood pressure readings taken on two or more office visits. Individuals older than 50 years are classified as having hypertension if their blood pressure is consistently at least 140 mmHg systolic or 90 mmHg diastolic. Patients with blood pressures higher than 130/80 mmHg with concomitant presence of diabetes mellitus or kidney disease require further treatment (Chobanian et al. (December 2003). “Seventh report of the Joint National Committee on Prevention, Detection, Evaluation, and Treatment of High Blood Pressure”. Hypertension 42(6): 1206-52.)

In some aspects, the subject suffers from a metabolic disease or metabolic syndrome. Metabolic Syndrome, also known as metabolic syndrome X, insulin resistance syndrome or Reaven's syndrome, is a disorder that affects over 50 million Americans. Metabolic Syndrome is typically characterized by a clustering of at least three or more of the following risk factors: (1) abdominal obesity (excessive fat tissue in and around the abdomen), (2) atherogenic dyslipidemia (blood fat disorders including high triglycerides, low HDL cholesterol and high LDL cholesterol that enhance the accumulation of plaque in the artery walls), (3) elevated blood pressure, (4) insulin resistance or glucose intolerance, (5) prothrombotic state (e.g., high fibrinogen or plasminogen activator inhibitor-1 in blood), and (6) pro-inflammatory state (e.g., elevated C-reactive protein in blood). Other risk factors may include aging, hormonal imbalance and genetic predisposition.

Metabolic Syndrome is associated with an increased the risk of coronary heart disease and other disorders related to the accumulation of vascular plaque, such as stroke and peripheral vascular disease, referred to as atherosclerotic cardiovascular disease (ASCVD). Patients with Metabolic Syndrome may progress from an insulin resistant state in its early stages to full blown type II diabetes with further increasing risk of ASCVD. Without intending to be bound by any particular theory, the relationship between insulin resistance, Metabolic Syndrome and vascular disease may involve one or more concurrent pathogenic mechanisms including impaired insulin-stimulated vasodilation, insulin resistance-associated reduction in NO availability due to enhanced oxidative stress, and abnormalities in adipocyte-derived hormones such as adiponectin (Lteif and Mather, Can. J. Cardiol. 20 (suppl. B):66B-76B (2004)).

According to the 2001 National Cholesterol Education Program Adult Treatment Panel (ATP III), any three of the following traits in the same individual meet the criteria for Metabolic Syndrome: (a) abdominal obesity (a waist circumference over 102 cm in men and over 88 cm in women); (b) serum triglycerides (150 mg/dl or above); (c) HDL cholesterol (40 mg/dl or lower in men and 50 mg/dl or lower in women); (d) blood pressure (130/85 or more); and (e) fasting blood glucose (110 mg/dl or above). According to the World Health Organization (WHO), an individual having high insulin levels (an elevated fasting blood glucose or an elevated post meal glucose alone) with at least two of the following criteria meets the criteria for Metabolic Syndrome: (a) abdominal obesity (waist to hip ratio of greater than 0.9, a body mass index of at least 30 kg/m2, or a waist measurement over 37 inches); (b) cholesterol panel showing a triglyceride level of at least 150 mg/dl or an HDL cholesterol lower than 35 mg/dl; (c) blood pressure of 140/90 or more, or on treatment for high blood pressure). (Mathur, Ruchi, “Metabolic Syndrome,” ed. Shiel, Jr., William C., MedicineNet.com, May 11, 2009).

For purposes herein, if an individual meets the criteria of either or both of the criteria set forth by the 2001 National Cholesterol Education Program Adult Treatment Panel or the WHO, that individual is considered as afflicted with Metabolic Syndrome.

With regard to the methods of the invention, in some embodiments, the subject suffers from diabetes or obesity or suffers from both diabetes and obesity.

In some embodiments, the subject does not suffer from a cardiac injury or a structural heart disease other than the diastolic dysfunction or heart failure being treated or prevented or diagnosed by the inventive method. By “cardiac injury” is meant a disruption of normal cardiac myocyte membrane integrity resulting in the loss into the extracellular space or intracellular constituents including detectable levels of biologically active cytosolic and structure proteins (e.g., troponin, creatine kinase, myoglobin, heart-type fatty acid binding protein, lactate dehydrogenase). By “structural heart disease” is meant any disease that affects the heart muscle or changes the architecture of the heart. In some aspects, the subject does not suffer from ischemic heart disease, chronic stable angina, chronic angina. In some aspects, the subject does not suffer from ischemia, ischemia-reperfusion or coronary artery occlusion-reperfusion, ischemic heart disease, myocardial injury, myocardial toxicity, myocardial infarction, congenital heart lesion, valvular stenosis or valvular regurgitation, coronary artery disease, chronic angina, chronic stable angina, arrhythmias. In some aspects, the subject does not suffer from a myocardial trauma, a myocardial toxicity, a viral infection, a deficiency in nutrients. In some aspects, the subject does not suffer from myocarditis.

In regards to the methods of treatment provided herein, the subject in some embodiments is a subject in need thereof. In some aspects, the subject is a subject suffering from diastolic dysfunction, e.g., any of the forms of diastolic dysfunction described in the section set forth herein entitled “Diastolic Dysfunction.” In some aspects, the subject is a subject suffering from heart failure, e.g., diastolic heart failure, heart failure with preserved ejection fraction, any of the types of heart failure described in the section set forth herein entitled “Heart Failure.”

Control subjects

In some embodiments, the control subject is a matched control of the same species, gender, ethnicity, age group, smoking status, BMI, current therapeutic regimen status, medical history, or a combination thereof, but differs from the subject being diagnosed in that the control does not suffer from diastolic dysfunction. In some embodiments, the control subject is a matched control of the same species, gender, ethnicity, age group, smoking status, BMI, current therapeutic regimen status, medical history, or a combination thereof, but differs from the subject being diagnosed in that the control subject suffers from neither diastolic dysfunction nor systolic dysfunction. In alternative aspects, the control subject is a matched control of the same species, gender, ethnicity, age group, smoking status, BMI, current therapeutic regimen status, medical history, or a combination thereof, but differs from the subject being diagnosed in that the control does not suffer from diastolic dysfunction but does suffer from systolic dysfunction.

With regard to the method of diagonising diastolic dysfunction in the absence of systolic dysfunction or the method of determining a therapeutic regimen for a subject, when the subject being diagnosed (e.g., the test subject) or the subject for which a therapeutic regimen is being determined is a subject exhibiting a sign or symptom of heart failure, the control subject in some embodiments is a control subject that also exhibits a sign or symptom of heart failure. In some aspects, the subject being diagnosed and the control subject are matched in that both exhibit the same signs and/or symptoms of heart failure. In further aspects, the control subject is a control subject which suffers from systolic dysfunction.

With regard to the method of diagnosing a type of heart failure, when the subject being diagnosed is a subject suffering from a heart failure, the control subject in some embodiments is a control subject suffering from heart failure. In some aspects, the control subject suffers from a systolic heart failure or a heart failure with reduced ejection fraction.

Pharmaceutically Acceptable Salts

With regard to the present disclosures, the therapeutic agent for treating diastolic dysfunction or the agent which increases adiponectin levels (collectively referred to hereinafter as “active agents”) in some aspects is in the form of a salt, e.g., a pharmaceutically acceptable salt. Such salts can be prepared in situ during the final isolation and purification of the active agent or separately prepared by reacting a free base function with a suitable acid. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate.

Basic addition salts also can be prepared in situ during the final isolation and purification of the active agent, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like.

Further, basic nitrogen-containing groups can be quaternized with such active agents as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

Conjugates

With regard to the present disclosures, in some aspects, the active agent is in the form of a conjugate, e.g., is conjugated to a heterologous moiety. As used herein, the term “heterologous moiety” is synonymous with the term “conjugate moiety” and refers to any molecule (chemical or biochemical, naturally-occurring or non-coded) which is different from the active agents described herein. Exemplary conjugate moieties that can be linked to any of the active agents described herein include but are not limited to a heterologous peptide or polypeptide (including for example, a plasma protein), a targeting agent, an immunoglobulin or portion thereof (e.g., variable region, CDR, or Fc region), a diagnostic label such as a radioisotope, fluorophore or enzymatic label, a polymer including water soluble polymers, or other therapeutic or diagnostic agents. In some embodiments a conjugate is provided comprising an active agent and a plasma protein, wherein the plasma protein is selected from the group consisting of albumin, transferin, fibrinogen and globulins. In some embodiments the plasma protein moiety of the conjugate is albumin or transferin. The conjugate in some embodiments comprises one or more of the active agents described herein and one or more of: a peptide, a polypeptide, a nucleic acid molecule, an antibody or fragment thereof, a polymer, a quantum dot, a small molecule, a toxin, a diagnostic agent, a carbohydrate, an amino acid, each of which are distinct from the active agents described herein.

In some embodiments, the heterologous moiety is a polymer. In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

In some aspects, the polymer is a biodegradable polymer, including a synthetic biodegradable polymer (e.g., polymers of lactic acid and glycolic acid, polyanhydrides, poly(ortho)esters, polyurethanes, poly(butic acid), poly(valeric acid), and poly(lactide-cocaprolactone)), and a natural biodegradable polymer (e.g., alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins (e.g., zein and other prolamines and hydrophobic proteins)), as well as any copolymer or mixture thereof. In general, these materials degrade either by enzymatic hydrolysis or exposure to water in vivo, by surface or bulk erosion.

In some aspects, the polymer is a bioadhesive polymer, such as a bioerodible hydrogel described by H. S. Sawhney, C. P. Pathak and J. A. Hubbell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In some embodiments, the polymer is a water-soluble polymer or a hydrophilic polymer. Hydrophilic polymers are further described herein under “Hydrophilic Moieties.” Suitable water-soluble polymers are known in the art and include, for example, polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof.

In specific embodiments, the polymer is a polyalkylene glycol, including, for example, polyethylene glycol (PEG).

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is attached via non-covalent or covalent bonding to the active agent of the present disclosure. In certain aspects, the heterologous moiety is attached to the active agent of the present disclosure via a linker. Linkage can be accomplished by covalent chemical bonds, physical forces such electrostatic, hydrogen, ionic, van der Waals, or hydrophobic or hydrophilic interactions. A variety of non-covalent coupling systems may be used, including biotin-avidin, ligand/receptor, enzyme/substrate, nucleic acid/nucleic acid binding protein, lipid/lipid binding protein, cellular adhesion molecule partners; or any binding partners or fragments thereof which have affinity for each other.

The active agent in some embodiments is linked to conjugate moieties via direct covalent linkage. In some embodiments, reactive groups on the active agent or conjugate moiety include, e.g., an aldehyde, amino, ester, thiol, α-haloacetyl, maleimido or hydrazino group. Derivatizing agents include, for example, maleimidobenzoyl sulfosuccinimide ester, N-hydroxysuccinimide (through lysine residues), glutaraldehyde, succinic anhydride or other agents known in the art. Alternatively, the conjugate moieties can be linked to the active agent indirectly through intermediate carriers, such as polysaccharide or polypeptide carriers. Examples of polysaccharide carriers include aminodextran. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier.

Carboxyl groups are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Another type of covalent modification involves chemically or enzymatically coupling glycosides to the active agent. Sugar(s) may be attached to free carboxyl groups, free sulfhydryl groups, free hydroxyl groups or an amide group. These methods are described in WO87/05330 published 11 Sep. 1987, and in Aplin and Wriston, CRC Crit. Rev. Biochem., pp. 259-306 (1981).

In some embodiments, the conjugate comprises a linker that joins the active agent to the heterologous moiety. In some aspects, the linker comprises a chain of atoms from 1 to about 60, or 1 to 30 atoms or longer, 2 to 5 atoms, 2 to 10 atoms, 5 to 10 atoms, or 10 to 20 atoms long. In some embodiments, the chain atoms are all carbon atoms. In some embodiments, the chain atoms in the backbone of the linker are selected from the group consisting of C, O, N, and S. Chain atoms and linkers may be selected according to their expected solubility (hydrophilicity) so as to provide a more soluble conjugate. In some embodiments, the linker provides a functional group that is subject to cleavage by an enzyme or other catalyst or hydrolytic conditions found in the target tissue or organ or cell. In some embodiments, the length of the linker is long enough to reduce the potential for steric hindrance. In some embodiments, the linker is an amino acid or a peptidyl linker. Such peptidyl linkers may be any length. Exemplary linkers are from about 1 to 50 amino acids in length, 5 to 50, 3 to 5, 5 to 10, 5 to 15, or 10 to 30 amino acids in length.

Conjugates: Hydrophilic Moieties

The active agents described herein can be further modified to improve its solubility and stability in aqueous solutions at physiological pH, while retaining its biological activity. Hydrophilic moieties such as PEG groups can be attached to the active agents under any suitable conditions known in the art, including, for example, via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, a-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., alpha-iodo acetic acid, alpha-bromoacetic acid, alpha-chloroacetic acid). If attached to the active agents by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

Suitable hydrophilic moieties include polyethylene glycol (PEG), polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1, 3-dioxolane, poly-1,3,6-trioxane, ethylene/maleic anhydride copolymer, poly (3-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof. Dextrans are polysaccharide polymers of glucose subunits, predominantly linked by α1-6 linkages. Dextran is available in many molecular weight ranges, e.g., about 1 kD to about 100 kD, or from about 5, 10, 15 or 20 kD to about 20, 30, 40, 50, 60, 70, 80 or 90 kD. Linear or branched polymers are contemplated. Resulting preparations of conjugates may be essentially monodisperse or polydisperse, and may have about 0.5, 0.7, 1, 1.2, 1.5 or 2 polymer moieties per compound.

Conjugates: Multimers

In some embodiments, the conjugate comprising the active agent is in the form of a multimer or dimer, including homo- or hetero- multimers or homo- or hetero- dimers. Two or more of the active agents can be linked together using standard linking agents and procedures known to those skilled in the art. In certain embodiments, the linker connecting the two (or more) analogs is PEG, e.g., a 5 kDa PEG, 20 kDa PEG. In some embodiments, the linker is a disulfide bond. For example, each monomer of the dimer may comprise a sulfhydryl and the sulfur atom of each participates in the formation of the disulfide bond.

Conjugates: Targeted Forms

One of ordinary skill in the art will readily appreciate that the active agents of the disclosure can be modified in any number of ways, such that the therapeutic or prophylactic efficacy of the active agent of the present disclosures is increased through the modification. For instance, the active agent of the present disclosure can be conjugated either directly or indirectly through a linker to a targeting moiety. The practice of conjugating compounds to targeting moieties is known in the art. See, for instance, Wadhwa et al., J Drug Targeting, 3, 111-127 (1995) and U.S. Pat. No. 5,087,616. The term “targeting moiety” as used herein, refers to any molecule or agent that specifically recognizes and binds to a cell-surface receptor, such that the targeting moiety directs the delivery of the active agent of the present disclosures to a population of cells on which surface the receptor is expressed. Targeting moieties include, but are not limited to, antibodies, or fragments thereof, peptides, hormones, growth factors, cytokines, and any other natural or non-natural ligands, which bind to cell surface receptors (e.g., Epithelial Growth Factor Receptor (EGFR), T-cell receptor (TCR), B-cell receptor (BCR), CD28, Platelet-derived Growth Factor Receptor (PDGF), nicotinic acetylcholine receptor (nAChR), etc.). As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups. The term “linker” in some embodiments refers to any agent or molecule that bridges the active agent of the present disclosures to the targeting moiety. One of ordinary skill in the art recognizes that sites on the active agent of the present disclosures, which are not necessary for the function of the active agent, are ideal sites for attaching a linker and/or a targeting moiety, provided that the linker and/or targeting moiety, once attached to the active agent, do(es) not interfere with the function of the active agent, i.e., the ability to treat diastolic dysfunction or increase adiponectin levels, as described herien.

Pharmaceutical Compositions and Formulations

In some embodiments, the active agent of the present disclosures, the pharmaceutically acceptable salt thereof, or the conjugate comprising the active agent, is formulated into a pharmaceutical composition comprising the active agent, the pharmaceutically acceptable salt thereof, or the conjugate comprising the active agent, along with a pharmaceutically acceptable carrier, diluent, or excipient.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. The pharmaceutical composition in some aspects comprises the active agent of the present disclosure at a concentration of at least A, wherein A is about 0.001 mg/ml, about 0.01 mg/ml, 0 about 1 mg/ml, about 0.5 mg/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml or higher. In some embodiments, the pharmaceutical composition comprises the active agent at a concentration of at most B, wherein B is about 30 mg/ml, about 25 mg/ml, about 24 mg/ml, about 23, mg/ml, about 22 mg/ml, about 21 mg/ml, about 20 mg/ml, about 19 mg/ml, about 18 mg/ml, about 17 mg/ml, about 16 mg/ml, about 15 mg/ml, about 14 mg/ml, about 13 mg/ml, about 12 mg/ml, about 11 mg/ml, about 10 mg/ml, about 9 mg/ml, about 8 mg/ml, about 7 mg/ml, about 6 mg/ml, about 5 mg/ml, about 4 mg/ml, about 3 mg/ml, about 2 mg/ml, about 1 mg/ml, or about 0.1 mg/ml. In some embodiments, the compositions may contain an active agent at a concentration range of A to B mg/ml, for example, about 0.001 to about 30.0 mg/ml.

Depending on the route of administration, the particular active agent intended for use, as well as other factors, the pharmaceutical composition may comprise additional pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

Accordingly, in some embodiments, the pharmaceutical composition comprises any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, a-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC)chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil, type II, hydroxyethyl cellulose, 2-hydroxyethyl-β-cyclodextrin, hydroxypropyl cellulose, low-substituted hydroxypropyl cellulose, 2-hydroxypropyl-13-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, postassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-β-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety, discloses various components used in formulating pharmaceutically acceptable compositions and known techniques for the preparation thereof. Except insofar as any conventional agent is incompatible with the pharmaceutical compositions, its use in pharmaceutical compositions is contemplated. Supplementary active ingredients also can be incorporated into the compositions.

In some embodiments, the foregoing component(s) may be present in the pharmaceutical composition at any concentration, such as, for example, at least A, wherein A is 0.0001% w/v, 0.001% w/v, 0.01% w/v, 0.1% w/v, 1% w/v, 2% w/v, 5% w/v, 10% w/v, 20% w/v, 30% w/v, 40% w/v, 50% w/v, 60% w/v, 70% w/v, 80% w/v, or 90% w/v. In some embodiments, the foregoing component(s) may be present in the pharmaceutical composition at any concentration, such as, for example, at most B, wherein B is 90% w/v, 80% w/v, 70% w/v, 60% w/v, 50% w/v, 40% w/v, 30% w/v, 20% w/v, 10% w/v, 5% w/v, 2% w/v, 1% w/v, 0.1% w/v, 0.001% w/v, or 0.0001%. In other embodiments, the foregoing component(s) may be present in the pharmaceutical composition at any concentration range, such as, for example from about A to about B. In some embodiments, A is 0.0001% and B is 90%.

The pharmaceutical compositions may be formulated to achieve a physiologically compatible pH. In some embodiments, the pH of the pharmaceutical composition may be at least 5, at least 5.5, at least 6, at least 6.5, at least 7, at least 7.5, at least 8, at least 8.5, at least 9, at least 9.5, at least 10, or at least 10.5 up to and including pH 11, depending on the formulation and route of administration. In certain embodiments, the pharmaceutical compositions may comprise buffering agents to achieve a physiological compatible pH. The buffering agents may include any compounds capabale of buffering at the desired pH such as, for example, phosphate buffers (e.g., PBS), triethanolamine, Tris, bicine, TAPS, tricine, HEPES, TES, MOPS, PIPES, cacodylate, MES, and others. In certain embodiments, the strength of the buffer is at least 0.5 mM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, at least 100 mM, at least 120 mM, at least 150 mM, or at least 200 mM. In some embodiments, the strength of the buffer is no more than 300 mM (e.g., at most 200 mM, at most 100 mM, at most 90 mM, at most 80 mM, at most 70 mM, at most 60 mM, at most 50 mM, at most 40 mM, at most 30 mM, at most 20 mM, at most 10 mM, at most 5 mM, at most 1 mM).

Routes of Administration

With regard to the invention, the active agent, pharmaceutical composition comprising the same, conjugate comprising the same, or pharmaceutically acceptable salt thereof, may be administered to the subject by any suitable route of administration. The following discussion on routes of administration is merely provided to illustrate exemplary embodiments and should not be construed as limiting the scope in any way.

Formulations suitable for oral administration can consist of (a) liquid solutions, such as an effective amount of the active agent of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the active agent of the present disclosure in a flavor, usually sucrose and acacia or tragacanth, as well as pastilles comprising the active agent of the present disclosure in an inert base, such as gelatin and glycerin, or sucrose and acacia, emulsions, gels, and the like containing, in addition to, such excipients as are known in the art.

The active agents of the present disclosure, alone or in combination with other suitable components, can be delivered via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the active agent is formulated into a powder blend or into microparticles or nanoparticles. Suitable pulmonary formulations are known in the art. See, e.g., Qian et al., Int J Pharm 366: 218-220 (2009); Adjei and Garren, Pharmaceutical Research, 7(6): 565-569 (1990); Kawashima et al., J Controlled Release 62(1-2): 279-287 (1999); Liu et al., Pharm Res 10(2): 228-232 (1993); International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2- dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl -imidazoline quaternary ammonium salts, and (e) mixtures thereof.

The parenteral formulations in some embodiments contain from about 0.5% to about 25% by weight of the active agent of the present disclosure in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the active agent of the present disclosures can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

It will be appreciated by one of skill in the art that, in addition to the above-described pharmaceutical compositions, the active agent of the disclosure can be formulated as inclusion complexes, such as cyclodextrin inclusion complexes, or liposomes.

Dosages

The active agents of the disclosure are believed to be useful in methods of treating a diastolic dysfunction, as well as related conditions, e.g., heart failure with preserved ejection fraction, as described herein. For purposes of the disclosure, the amount or dose of the active agent administered should be sufficient to effect, e.g., a therapeutic or prophylactic response, in the subject or animal over a reasonable time frame. For example, the dose of the active agent of the present disclosure should be sufficient to treat diastolic dysfunction or heart failure with preserved ejection fraction as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which diastolic dysfunction is treated upon administration of a given dose of the active agent of the present disclosure to a mammal among a set of mammals, each set of which is given a different dose of the active agent, could be used to determine a starting dose to be administered to a mammal. The extent to which diastolic dysfunction is treated upon administration of a certain dose can be assayed by methods known in the art, including, for instance, the methods described in the EXAMPLES set forth below.

The dose of the active agent of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the invention, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

In some embodiments, the active agent is formulated for injection, is a compound of Formula II, e.g., ranolazine, and is administered to the subject at a dose between about 1 and about 20 mg/kg body weight of the subject for an injection, (e.g., between about 5 and about 15 mg/kg, between about 10 to about 12 mg/kg, about 9 mg/kg, about 10 mg/kg, about 11 mg/kg, about 12 mg/kg). In some embodiments, the active agent is formulated for infusion (e.g., intravenous infusion), is a compound of Formula II, e.g., ranolazine, and is administered at a dose between about 1 mg/kg/h to about 20 mg/kg/h (e.g., about 1 mg/kg/h, about 2 mg/kg/h, about 3 mg/kg/h, about 4 mg/kg/h, about 5 mg/kg/h, about 6 mg/kg/h, about 7 mg/kg/h, about 8 mg/kg/h, about 9 mg/kg/h, about 10 mg/kg/h, about 11 mg/kg/h, about 12 mg/kg/h, about 13 mg/kg/h, about 14 mg/kg/h, about 15 mg/kg/h, about 16 mg/kg/h, about 17 mg/kg/h, about 18 mg/kg/h, about 19 mg/kg/h, about 20 mg/kg/h)

In some embodiments, wherein the active agent is formulated for oral administration and is a compound of Formula II, e.g., ranolazine, the dose administered to the subject is between about 100 and about 2000 mg (e.g., about 100 mg, about 200 mg, about 250 mg, about 300 mg, about 400 mg, about 500 mg, about 600 mg, about 700 mg, about 750 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg). In some aspects, the oral dosage is administered once daily, twice daily, three times daily, or four times daily.

In some embodiments, the dosage of the active agent is any of the above dosages, but the active agent is other than a compound of Formula II (e.g., ranolazine).

In some embodiments, the administered dose of the active agent (e.g., any of the doses described above), provides the subject with a plasma concentration of the active agent of at least or about 500 nM. In some aspects, the administered dose of the active agent provides the subject with a plasma concentration of the active agent within a range of about 500 nM to about 2500 nM (e.g., about 750 nM to about 2000 nM, about 1000 nM to about 1500 nM). In some aspects, the dose of the active agent provides the subject with a plasma concentration of the active agent which is below 100 μmol/L, e.g., below 50 μmol/L, below 25 μmol/L, below 10 μmol/L.

Controlled Release Formulations

In some embodiments, the active agents described herein can be modified into a depot form, such that the manner in which the active agent of the present disclosures is released into the body to which it is administered is controlled with respect to time and location within the body (see, for example, U.S. Pat. No. 4,450,150). Depot forms of active agents of the present disclosures can be, for example, an implantable composition comprising the active agents and a porous or non-porous material, such as a polymer, wherein the active agent is encapsulated by or diffused throughout the material and/or degradation of the non-porous material. The depot is then implanted into the desired location within the body of the subject and the active agent is released from the implant at a predetermined rate.

The pharmaceutical composition comprising the active agent in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The instant compositions may further comprise, for example, micelles or liposomes, or some other encapsulated form, or may be administered in an extended release form to provide a prolonged storage and/or delivery effect. The disclosed pharmaceutical formulations may be administered according to any regime including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly.

Combinations

In some embodiments, the active agents described herein are administered alone, and in alternative embodiments, the active agents described herein are administered in combination with another therapeutic agent which aims to treat or prevent any of the diseases or medical conditions described herein, e.g., diastolic dysfunction. In exemplary embodiments, an active agent of a first structure is co-administered with (simultaneously or sequentially) another active agent of different structure. In alternative or additional embodiments, the active agents described herein may be co-administered with (simultaneously or sequentially) a therapeutic agent for the treatment of hypertension, including, for example, a thiazide diuretic (e.g., chlorothiazine, hydrochlorothiazide, metolazone), a beta blocker (a.k.a, beta-adrenergic blocking agent (e.g., acebutolol, atenolol, bisoprolol, carvedilol, metoprolol, nadolol, nebivolol, penbutolol, propranolol)), an angiotensin-convertine enzyme (ACE) inhibitor (e.g., benazepril, captopril, enalapril, fosinopril, lisinopril, moexipril, perindopril, quinapril, ramipril, trandolarpil), an angiotensin II receptor blocker (a.k.a., ARBs (e.g., candesartan, eprosartan, irbesartan, losartan, olmesartan, telmisartan, valsartan)), a calcium channel blocker (a.k.a., calcium antagonist, (e.g., amlodipine, diltiazem, felodipine, isradipine, nicardipine, nifedipine, nisoldipine, verapramil)), a rennin inhibitor (e.g., Aliskiren), an alpha blocker (a.k.a, an alpha-adrenergic antagonist, alpha-adrenergic blocking agent, adrenergic blocking agent, alpha-blocking agent, (e.g., doxazosin, prazosin, terazosin, tamsulosin, alfuzosin)), an alpha-beta blocker (a.k.a, alpha-beta adrenergic blocker (e.g., carvedilol, labetalol), a central-acting agent (a.k.a., central adrenergic inhibitor, central alpha agonist, central agonist, (e.g., clonidine, guanfacine, methyldopa)), a vasodilator (e.g., hydralazine, minoxidil).

In alternative or additional embodiments, the active agent is co-administered with (simultaneously or sequentially) a therapeutic agent for the treatment of diabetes or obesity. Anti-diabetic agents known in the art or under investigation include insulin, leptin, Peptide YY (PYY), Pancreatic Peptide (PP), fibroblast growth factor 21 (FGF21), Y2Y4 receptor agonists, sulfonylureas, such as tolbutamide (Orinase), acetohexamide (Dymelor), tolazamide (Tolinase), chlorpropamide (Diabinese), glipizide (Glucotrol), glyburide (Diabeta, Micronase, Glynase), glimepiride (Amaryl), or gliclazide (Diamicron); meglitinides, such as repaglinide (Prandin) or nateglinide (Starlix); biguanides such as metformin (Glucophage) or phenformin; thiazolidinediones such as rosiglitazone (Avandia), pioglitazone (Actos), or troglitazone (Rezulin), or other PPARy inhibitors; alpha glucosidase inhibitors that inhibit carbohydrate digestion, such as miglitol (Glyset), acarbose (Precose/Glucobay); exenatide (Byetta) or pramlintide; Dipeptidyl peptidase-4 (DPP-4) inhibitors such as vildagliptin or sitagliptin; SGLT (sodium-dependent glucose transporter 1) inhibitors; glucokinase activators (GKA); glucagon receptor antagonists (GRA); or FBPase (fructose 1,6-bisphosphatase) inhibitors, GLP-1 agonists.

Anti-obesity agents known in the art or under investigation include appetite suppressants, including phenethylamine type stimulants, phentermine (optionally with fenfluramine or dexfenfluramine), diethylpropion (Tenuate®), phendimetrazine (Prelu-2®, Bontril®), benzphetamine (Didrex®), sibutramine (Meridia®, Reductil®); rimonabant (Acomplia®), other cannabinoid receptor antagonists; oxyntomodulin; fluoxetine hydrochloride (Prozac); Qnexa (topiramate and phentermine), Excalia (bupropion and zonisamide) or Contrave (bupropion and naltrexone); or lipase inhibitors, similar to XENICAL (Orlistat) or Cetilistat (also known as ATL-962), or GT 389-255.

In some embodiments, the active agent is administered in combination with aspirin, or other therapeutic agent which promotes cardiac efficiency.

In view of the foregoing, the invention further provides pharmaceutical compositions and kits additionally comprising one of these other therapeutic agents in combination with the active agent. The additional therapeutic agent may be administered simultaneously or sequentially with the active agent of the present disclosure. In some aspects, the active agent is administered before the additional therapeutic agent, while in other aspects, the active agent is administered after the additional therapeutic agent.

Kits

Provided herein are kits, e.g., diagnostic kits, comprising a binding agent or substrate specific for a positive RAS marker, binding agent or substrate specific for a negative RAS marker, binding agent or substrate specific for a positive oxidative stress marker, binding agent or substrate specific for a negative oxidative stress marker, a binding agent specific for adiponectin, or a combination thereof. As used herein, the term “binding agent” refers to any compound which specifically binds to the RAS marker (e.g., positive RAS marker, negative RAS marker), oxidative stress marker (positive oxidative stress marker, negative oxidative stress marker), or adiponectin. In some aspects, the binding agent is an antibody, antigen binding fragment, an aptamer, a peptide, or a nucleic acid probe. In some aspects, the RAS marker is any of those described herein. In some aspects, the oxidative stress marker is any of those described herein. In some aspects, the substrate is a peptide, a protein, a nucleic acid molecule, a lipid, a carbohydrate, a sugar, an amino acid, or a small molecule.

In some aspects, the kit comprises a collection of nucleic acid probes which specifically bind to genes which activate in response to RAS or oxidative stress. In some aspects, the collection of nucleic acid probes is formatted in an array on a solid support, e.g., a gene chip.

In some aspects, the kit comprises a collection of antibodies which specifically bind to a combination of: one or more positive RAS markers, one or more negative RAS markers, one or more positive oxidative stress markers, one or more negative oxidative stress markers, and adiponectin. In some aspects, the kit comprises a multi-well microtiter plate, wherein each well comprises an antibody having a specificity which is unique to the antibodies of the other wells.

In some aspects, the kit comprises a collection of substrates which specifically react with a combination of: one or more positive RAS markers, one or more negative RAS markers, one or more positive oxidative stress markers, one or more negative oxidative stress markers, and adiponectin. In some aspects, the kit comprises a multi-well microtiter plate, wherein each well comprises a substrate having a specificity which is unique to the substrates of the other wells.

In some aspects, the kits further comprises instructions for use, e.g., instructions for diagnosing diastolic dysfunction in the absence of systolic dysfunction. In some aspects, the instructions are provided as a paper copy of instructions, an electronic copy of instructions, e.g., a compact disc, a flash drive, or other electronic medium. In some aspects, the instructions are provided by way of providing directions to an internet site at which the instructions may be accessed by the user.

In some aspects, the instructions comprise a step in which the user compares data relating to a positive RAS marker, a negative RAS marker, a positive oxidative stress marker, a negative oxidative stress marker, an adiponectin level, or a combination thereof, to a database containing correlation data between the data and to a diagnosis of diastolic dysfunction in the absence of systolic dysfunction. In some aspects, the kit comprises an electronic copy of a database containing correlation data between the data relating to a positive RAS marker, a negative RAS marker, a positive oxidative stress marker, a negative oxidative stress marker, an adiponectin level, or a combination thereof, and to a diagnosis of diastolic dysfunction in the absence of systolic dysfunction. In some aspects, the kit comprises an electronic copy of a computer software program which allows the user to compare the evidence of RAS activation, evidence of oxidative stress, or level of adiponectin with that of a control subject.

In alternative aspects, the instructions comprise a step in which the user provides data relating to a positive RAS marker, a negative RAS marker, a positive oxidative stress marker, a negative oxidative stress marker, an adiponectin level, or a combination thereof, to a provider and the provider, after analyzing the data, provides diagnostic information to the user.

In some aspects, the kits further comprise a unit for a collecting a sample, e.g., any of the samples described herein, of the subject. In some aspects, the unit for collecting a sample is a vial, a beaker, a tube, a microtiter plate, a petri dish, and the like.

The following examples are given merely to illustrate the present invention and not in any way to limit its scope.

### EXAMPLES

### Example 1

This example provides methods of assessing diastolic dysfunction in a mammal.

Noninvasive assessment of diastolic dysfunction: Mice were anesthetized, maintained at 37° C., and studied by echocardiography (Vevo 770, VisualSonics Inc, Toronto, Canada). M-mode images in the parasternal long axis and the left ventricle (LV) short-axis views at the mid-papillary level. Measurements were averaged from three consecutive beats during expiration. LV inflow velocities (E and A waves) were interrogated by conventional pulsed-wave Doppler from the apical four-chamber view. The mitral annulus longitudinal velocities (Sm, E′, and A′) were determined by pulsed-wave tissue Doppler from the apical four-chamber view. Interpretation was done by two investigators blinded to the treatment groups. First, baseline images were acquired. Subsequently, the mice were injected with 30 mg/kg ranolazine by intraperitoneal route, followed by a second echocardiogram 30 min later.

Invasive assessment of diastolic dysfunction: Mice were anesthetized with 1-1.5% isoflurane and maintained at 37° C. The pressure-volume (PV) catheter was inserted into the right common carotid artery and advanced into the LV. Inferior vena cava occlusion was performed via a midline abdominal incision. Volume and parallel conductance calibration were performed as previously described (Silberman et al., Circulation. 2010;121(4):519-528) Baseline hemodynamic measurements were obtained, and subsequently, the mice received an intravenous injection of ranolazine (5 mg/kg) followed by an infusion at 4.8 mg/kg/h, while additional hemodynamic measurements were recorded. Blood samples were obtained during the last five minutes of the procedure to determine the plasma ranolazine concentration.

### Example 2

The following materials and methods were used in the study described in this example:

Study Design and Patient Recruitment

In a cross-sectional, case-control study, 50 subjects with NYHA Class I-II HF symptoms and echocardiographic evidence of early DD, as defined by preserved left ventricle (LV) EF of >50% and abnormal echocardiographic LV relaxation pattern on pulsed-wave and tissue Doppler, and matched controls were recruited from the outpatient clinics and hospital at the Atlanta Veterans Affairs Medical Center and Emory University Hospital from July 2006 to February 2008 (www.clinicaltrials.gov; NCT00142194). Cases and controls were matched for age in decades, smoking history, and diabetes mellitus, known confounders in oxidative stress measurements. The protocol was approved by the Emory University Institutional Review Board.

Eligibility criteria for both groups included age >18 years, an echocardiogram with mitral valve inflow velocities and tissue Doppler measurements within six months of enrollment, normal sinus rhythm, LV EF between 50 and 70%, and normal systolic and diastolic cardiac dimensions on qualifying echocardiogram. Exclusion criteria included systemic inflammatory disease, malignant neoplasm, severe valvular heart disease, HF NYHA Class III or IV, untreated hyper- or hypothyroidism, greater than mild cardiac hypertrophy, cardiomyopathy of any etiology, blood pressure (BP) >180/100 mmHg on medications, any concurrent illness resulting in life expectancy <1 year, and current illicit drug or alcohol abuse. A written informed consent was obtained from all participants.

Clinical Data

Demographic and clinical data were collected from review of medical records, history and physical examination upon enrollment, and the qualifying echocardiogram. A single blood draw in a non-fasting state was obtained between 8:30 AM and 5:00 PM. Blood samples were collected from the antecubital vein and, for thiol measures, were immediately transferred to a microcentrifuge tube with 0.5 mL preservative solution of 100 mmol/L serine borate (pH 8.5), containing (per mL) 0.5 mg sodium heparin, 1 mg bathophenanthroline disulfonate sodium salt and 2 mg iodoacetic acid. Samples were analyzed at the Emory Biomarkers Core Laboratory.

Echocardiographic Data

Early DD was defined by impaired ventricular filling as evidenced by the ratio of peak velocity of blood across the mitral valve in early diastolic filling, the E wave, to that during atrial contraction, the A wave (E/A ratio <1), and the ratio of peak early (E′) and late (A′) mitral annular velocities recorded by conventional pulsed wave Doppler method (E′/A′ ≦1).¹² An independent cardiologist interpreted the studies using standard protocols.'¹²

Measurement of Oxidative Stress Markers

Markers used to measure systemic oxidative stress were the same as those we have characterized previously¹¹, redox potential of the ratios of oxidized to reduced glutathione (Eₕ GSH) and cysteine (Eₕ CyS) in plasma (thiol ratios)^{8,11}, DROMs⁹ and IsoPs.¹⁰ The samples were stored at −80° C. Samples from cases and controls were treated identically. Laboratory technicians were blinded to the clinical data. The redox states (Eₕ) of thiol/disulfide pools were calculated using Nernst equation:

Eₕ=Eo+RT/nF In [disulfide]/[thiol]²,

where Eo is the standard potential for redox couple, R is the gas constant, T is the absolute temperature, n is number of electrons transferred, and F is Faraday constant. Eo used for glutathione and cysteine redox couples was −264 mV and −250 mV, respectively. Less negative Eₕ numbers implied a more oxidized state.

For measurement of IsoPs, samples were acidified and a deuterated standard was added. This was followed by C-18 and Silica Sep-Pak extraction.¹⁰ IsoPs were then converted to pentafluorobenzyl esters which were subjected to thin layer chromatography. F2-IsoPs were quantified by gas chromatography/mass spectrometry by using an Agilent 5973 MS with computer interference. After dissolution of serum in acidic buffer, an additive (N-N-diethyl-para-phenylendiamine) was added for DROM measurements.⁹ Concentration of DROMs was determined through spectrometry (505 nm).

Measurement of RAS Activation

ACE activity and protein levels were analyzed in 31 (15 cases and 16 controls) subjects not taking any form of RAS inhibitor, since these are known to alter the measures.¹³ Heparinized human plasma (20-40 μL) was diluted 1:5 parts with phosphate buffered saline (PBS) and incubated at 37° C. with 200 μL of substrate for 2 hours. ACE activity was determined fluorimetrically with two different substrates, Hip-His-Leu (HHL, 5 mM) and Z-Phe-His-Leu (ZPHL, 2 mM) and expressed as mU/ml.¹⁴ Levels of ACE protein were determined using plate precipitation assay based on monoclonal antibody to the epitope localized on the N domain of ACE (9B9) and expressed as a percentage (%) of gold standard from pooled human plasma.^{14,15}

Western blot analysis was used to measure extracellular copper-zinc superoxide dismutase (ec-SOD) and the copper-delivering protein, ceruloplasmin (Cp), expression in representative samples from both groups. Briefly, plasma ecSOD or Cp was concentrated by Concanavalin-A sepharose chromatography, and protein expression was examined by immunoblotting with antibody against ecSOD or ceruloplasmin (Dako Cytomation, Carpinteria, Calif.).¹⁶

Statistical Analysis

Statistical analyses were performed using SAS software 9.1 (SAS Institute, Inc.). Sample size was based on a 0.90 power to detect the same difference that we observed in the least sensitive measure of oxidative stress in our previous study using a two-tailed α-level of 0.05.¹¹ Baseline characteristics with normal distribution were compared between cases and controls using a paired t-test for continuous variables and Chi-square/Fisher exact test for categorical variables. Non-parametric tests were used for variables with skewed distribution. Mean (and median, where appropriate) levels of oxidative stress and ACE markers in cases and controls were compared using t-test for normally distributed variables and NPARlWAY procedure (SAS software 9.1) for variables with skewed distribution. All variables significant on univariate analysis were entered into multiple logistic regression models to calculate adjusted odds ratios.

The following results were obtained from the study described in this example:

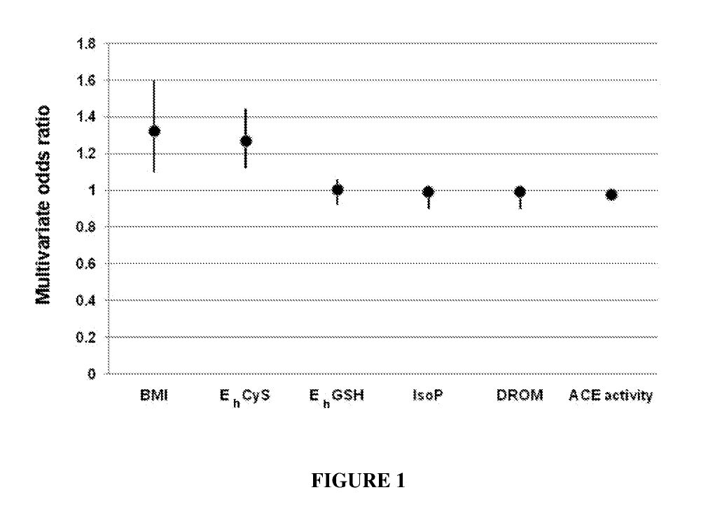

Fifty 50 patients with and without echocardiographic evidence of early DD were enrolled. The groups were well matched for known confounders in measurement of oxidative stress markers, including age (p=0.96), smoking (p=1.00) and diabetes mellitus (p=0.77). The mean age of cases and controls was 64.8±10.8 years (range: 45-83 years) and 65.0±11.3 years (range: 43-88 years), respectively. Univariate analysis showed that male sex (18 (72%) versus 11 (44%), p=0.04) and a higher mean BMI (29.6±4.8 versus 25.3±4.7, p=0.003) were the only two baseline variables associated with early DD. The cases and controls were statistically similar in all other baseline variables including race, hypertension, mean systolic BP, mean diastolic BP, hypercholesterolemia, use of different classes of antihypertensive agents and statins (Table I). The association of higher BMI with DD was maintained on a multivariate analysis (model 1) using all demographic and clinical parameters as predictive variables (adjusted OR: 1.3; 95% CI: 1.1-1.6; FIG. 1).

TABLE 1
Baseline characteristics of patients with
and without diastolic dysfunction

| | DD (N = 25) | Control (N = 25) | P-value |
|---|---|---|---|
| Demographic variables | | | |
| Age | 64.8 ± 10.8 | 65.0 ± 11.3 | 1 |
| Gender | | | 0.04 |
| Females | 7 (28%) | 14 (56%) | |
| Males | 18 (72%) | 11 (44%) | |
| Race* | | | 0.8 |
| White | 13 (54%) | 12 (48%) | |
| Black | 11 (46%) | 12 (48%) | |
| Clinical variables | | | |
| Smoking | 10 (40%) | 10 (40%) | 1 |
| Diabetes | 8 (32%) | 9 (36%) | 1 |
| BMI | 29.6 ± 4.8 | 25.3 ± 4.7 | 0.003 |
| Hypertension | 16 (64%) | 14 (56%) | 0.6 |
| Mean SBP (mm Hg) | 135.4 ± 19.2 | 127.4 ± 16.7 | 0.1 |
| Mean DBP (mm Hg) | 75.8 ± 13.0 | 74.0 ± 10.3 | 0.6 |
| Hypercholesteremia | 14 (56%) | 10 (40%) | 0.4 |
| Medications | | | |
| Beta Blocker | 15 (60%) | 10 (40%) | 0.3 |
| ACEI | 12 (48%) | 9 (36%) | 0.6 |
| ARB | 4 (16%) | 1 (4%) | 0.4 |
| Diuretic | 4 (16%) | 8 (32%) | 0.3 |
| Statin | 14 (56%) | 16 (64%) | 0.8 |

*1 (4%) Asian in each group;
DD, Diastolic dysfunction;
BMI, Body mass index;
SBP, Systolic blood pressure;
DBP, Diastolic blood pressure;
ACEI, Angiotensin converting enzyme inhibitor;
ARB, Angiotensin II receptor blocker.

Table 2 compares markers for oxidative stress, ACE activity and ACE protein levels in patients with and without DD.

TABLE 2
Oxidative stress markers and ACE in patients with and without diastolic dysfunction

| | DD | | Control | | |
|---|---|---|---|---|---|
| | Mean ± SD | Median | Mean ± SD | Median | P-value |
| Oxidative stress measures | | | | | |
| (Eₕ) CyS* | 70.1 ± 7.8 | 72.9 | 50.3 ± 11.6 | 50.6 | 0.001 |
| (Eₕ) GSH* | 118.8 ± 14.0 | 114.6 | 118.4 ± 16.5 | 117 | 0.9 |
| DROMs^{±} | 375.2 ± 132.4 | 341.3 | 474.5 ± 167.5 | 462.1 | 0.02 |
| IsoP [symbol] | 15 × 10² ± 6 × 10² | 13 × 10² | 73 × 10² ± 32 × 10² | 23 × 10² | 0.03 |
| ACE measures | | | | | |
| ACE-HHL^{‡} | 42.6 ± 9.6 | 40.2 | 36.8 ± 9 | 37.5 | 0.1 |
| ACE-ZPHL^{‡} | 39.0 ± 8.7 | 40.3 | 36.2 ± 8.9 | 36.7 | 0.4 |
| ACE PROT^{§} | 134.5 ± 38.2 | 112.8 | 101.9 ± 22.2 | 104.3 | 0.03 |

*mV;
^{±}Carr units; [symbol]
pg/ml;
^{‡}mU/ml;
^{§}percentage (%);
DD, Diastolic dysfunction;
Eₕ CyS, Redox potential of reduced to oxidized cysteine (negative);
Eₕ GSH, Redox potential of reduced to oxidized glutathione (negative);
DROM, Derivatives of reactive oxygen metabolites;
IsoP, Isoprostanes;
ACE-HHL, angiotensin converting enzyme activity measured using Hip-His-Leu (HHL) substrate;
ACE-ZPHL, angiotensin converting enzyme activity measured using Z-Phe-His-Leu (ZPHL) substrate;
ACE PROT, angiotensin converting enzyme protein levels.

Contrary to expectation, three of four oxidative stress measures suggested that early DD was associated with a reduced systemic state as compared to controls. Eₕ CyS was significantly more reduced (more negative) in patients with early DD (mean, −70.1±7.8 mV; median, −72.9 mV in cases versus mean, −50.3±11.6 mV; median, −50.6 mV in controls, p<0.001). There was no significant difference in Eₕ GSH (mean, −118.8±14.0 mV; median, −114.6 mV in cases versus mean, −118.4±16.5 mV; median, −117.0 mV in controls, p=0.93), a less sensitive measure of plasma redox state (23). IsoPs levels (mean, 1495±663 pg/mL; median, 1345 pg/mL in cases versus mean, 7385±3241 pg/mL; median, 2341 pg/mL in controls, p=0.03) and DROMs (mean, 375.2±132.4 Carr units; median, 341.3 Carr units in cases versus mean, 474.5±167.5 Carr units; median, 462.1 Can units in controls, p=0.02) were significantly lower in patients with DD. The association between a more reduced Eₕ CyS and DD was maintained on a multivariate analysis (model 2) using gender, BMI, and oxidative stress measures as predictive variables (adjusted OR: 1.22; 95% CI: 1.08-1.37).

ACE activity, determined with HHL as substrate, demonstrated a mild but statistically insignificant increase in the DD group compared to controls (mean, 42.6 ±9.6 mU/m1; median, 40.2 mU/ml in cases versus mean, 36.8±9.0 mU/ml; median, 37.5 mU/ml in controls, p=0.1). ACE protein levels were only marginally higher in patients with early DD (mean, 134.5±38.2%; median, 112.8% in cases versus mean, 101.9±22.2%; median, 104.3%; p=0.03; adjusted OR: 1.05; 95% CI: 1.01-1.09).

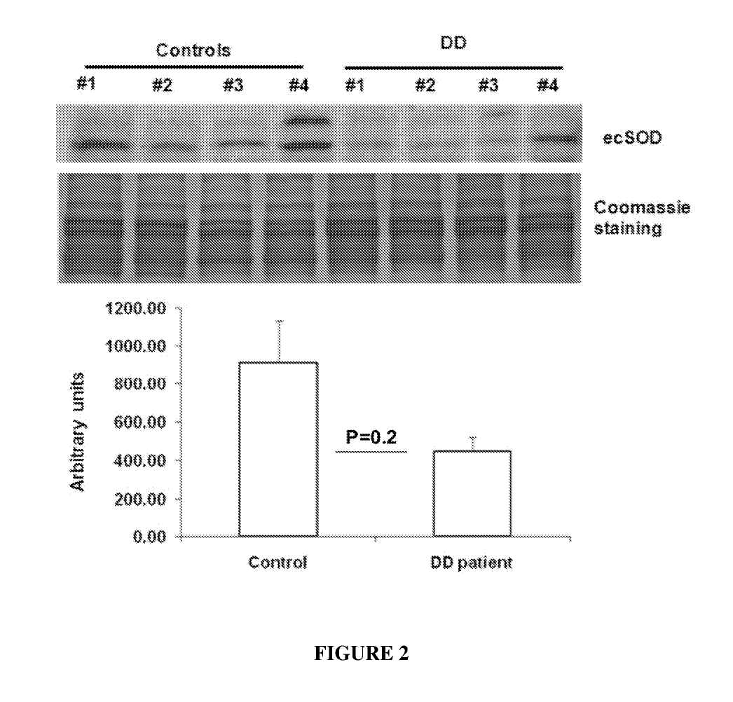

ec-SOD in representative samples from both groups was measured. Though there was mild decrease in ec-SOD activity in the DD group, this did not reach statistical significance (p=0.2). (FIG. 2) Since ecSOD is a copper enzyme, serum Cp, a marker protein for systemic copper, was also measured in the same samples but was not found to be altered in DD patients.

It has been proposed that RAS and subsequent oxidation play a role in pathogenesis of DD. The cardiovascular effects of Ang II are believed to be because of its activation of NADPH oxidase.⁴ Ang II also induces mitochondrial dysfunction, generating ROS such as superoxide (O₂^{.—}). Overall, these are thought to lead to a reduction in NO bioavailability and a defect in myocardial relaxation.¹⁷ We measured systemic oxidative stress markers and ACE activity in subjects with and without early DD.

We found no evidence of significant RAS activation or systemic oxidative stress in subjects with early DD when compared to a matched control group. Plasma ec-SOD activity and its copper delivering protein, Cp, were not raised in DD. This also suggests a lack of increase in ROS, since systemic oxidative stress is known to upregulate peripheral ec-SOD activity.²¹

In conclusion, we did not find evidence of systemic RAS activation or oxidative stress in patients with early DD. The lack of RAS activation and systemic oxidation seems to differentiate systolic from diastolic HF. This suggests different mechanisms in genesis or propagation of these two forms of HF, which would explain the difference in benefit with treatment modalities.

### Example 3

The following materials and methods were used in the study described in this example:

In a cross-sectional, case-control design, 25 subjects with NYHA Class I-II HF symptoms and echocardiographic evidence of early DD, and 25 age-matched controls were recruited from the outpatient clinics at the Atlanta Veterans Affairs Medical Center and Emory University Hospital. DD was defined by preserved left ventricle (LV) EF of >50% and abnormal echocardiographic parameters consistent with diastolic dysfunction ²¹ on pulsed-wave and tissue Doppler studies. The protocol was approved by the Emory University Institutional Review Board.

Eligibility criteria for both groups included age >18 years, an echocardiogram with mitral valve inflow velocities and tissue Doppler measurements within six months of enrollment, normal sinus rhythm, LV EF between 50 and 70%, and normal systolic and diastolic cardiac dimensions on qualifying echocardiogram. Exclusion criteria included systemic inflammatory disease, malignant neoplasm, severe valvular heart disease, HF NYHA Class III or IV, untreated hyper- or hypothyroidism, greater than mild cardiac hypertrophy, cardiomyopathy of any etiology, blood pressure (BP) >180/100 mmHg on medications, any concurrent illness resulting in life expectancy <1 year, and current illicit drug or alcohol abuse.

Demographic and clinical data were collected from review of medical records, history and physical examination upon enrollment, and the qualifying echocardiogram. A single blood draw was obtained from the antecubital vein from subjects in both groups. Samples were immediately transferred to a microcentrifuge tube with 0.5 mL preservative solution of 100 mmol/L serine borate (pH 8.5), containing (per mL) 0.5 mg sodium heparin, 1 mg bathophenanthroline disulfonate sodium salt and 2 mg iodoacetic acid to enable storage at
80 C. Plasma adiponectin was quantitatively determined using multimeric ELISA assay (ALPCO diagnostics, Salem, N.H.). Briefly, sample were divided into aliquots to assay for the two forms, total and HMW prior to pretreatment. To measure the total adiponectin, the sample was pretreated with sample pretreatment buffer (Citrate+sodium dodecyl sulfate). For quantification of HMW form, pretreatment was performed with Protease II, which selectively digests the MMW and LMW forms. Pretreated samples then were diluted with buffer in 1:101 dilutions. Then, samples were incubated with biotin labeled monoclonal antibody to human adiponectin for one hour. The sample was washed, enzyme labeled Streptavidin was added, and the plate was further incubated for 30 min. The plate was washed again and 50 μL of substrate solution was added and incubated for further 10 min at room temperature followed by the addition of Stop Reagent. After about 10-30 min, absorbance of each well was measured using a microplate reader set at 492 nm. Calibration curves were constructed from standards, and the concentration for the diluted samples was read from them by multiplying by the dilution factor.

Statistical analyses were performed using SPSS version 16 (SPSS Inc, Chicago, Ill.). Categorical data are presented as numbers (%). Continuous data with normal distribution are presented as mean ±standard deviation (SD) while those with a skewed distribution are presented as median (IR, interquartile range). General linear models were used to evaluate univariate association of baseline variables with DD. Linear regression analysis was performed to evaluate the independent association of predictive variables with DD. Linear regression model 1 evaluated the independent association of baseline variables found to be significant on univariate analysis. Linear regression model 2 evaluated the independent association of adiponectin or its fractions using age, gender and BMI as predictive co-variates. All investigators had direct access to the primary data.

The following results were obtained from the study described in this example:

The baseline demographic and clinical characteristics of patients with early DD and their age-matched controls are shown in Table 3.

TABLE 3
Baseline characteristics of patients with
and without diastolic dysfunction

| | DD (N = 25) | Control (N = 25) | P-value |
|---|---|---|---|
| Demographic variables | | | |
| Age | 64.8 ± 10.8 | 65.0 ± 11.3 | 0.9 |
| Gender | | | 0.04 |
| Females | 7 (28%) | 14 (56%) | |
| Males | 18 (72%) | 11 (44%) | |
| Race | | | 0.8 |
| White | 13 (54%) | 12 (48%) | |
| Black | 11 (46%) | 12 (48%) | |
| Clinical variables | | | |
| Smoking | 10 (40%) | 10 (40%) | 1.0 |
| Diabetes | 8 (32%) | 9 (36%) | 0.8 |
| BM I | 29.6 ± 4.8 | 25.3 ± 4.7 | 0.003 |
| Hypertension | 16 (64%) | 14 (56%) | 0.6 |
| Mean SBP (mm | 135.4 ± 19.2 | 127.4 ± 16.7 | 0.1 |
| Mean DBP (mm | 75.8 ± 13.0 | 74.0 ± 10.3 | 0.6 |
| Hypercholesteremi | 14 (56%) | 10 (40%) | 0.4 |
| Medications | | | |
| 13 Blocker | 15 (60%) | 10 (40%) | 0.3 |
| ACEI | 12 (48%) | 9 (36%) | 0.6 |
| ARB | 4 (16%) | 1 (4%) | 0.4 |
| Diuretic | 4 (16%) | 8 (32%) | 0.3 |
| Statin | 14 (56%) | 16 (64%) | 0.8 |

*1 (4%) Asian in each group;
DD, Diastolic dysfunction;
BMI, Body mass index;
SBP, Systolic blood pressure;
DBP. Diastolic blood pressure;
ACEI, Angiotensin converting enzyme inhibitor;
ARB, Angiotensin II receptor blocker.

Univariate analysis using general linear models showed that male sex (p=0.04) and a higher mean BMI (p=0.003) were the only two baseline variables associated with early DD (Table 3). There was no association of DD with race, hypertension, hypercholesterolemia, use of different classes of antihypertensive agents, or the use of statins. Only BMI retained significant association with DD in a linear regression analysis using variables significant on univariate analysis as covariates (Model 1; r²=0.46, t score=3.4, p=0.006).

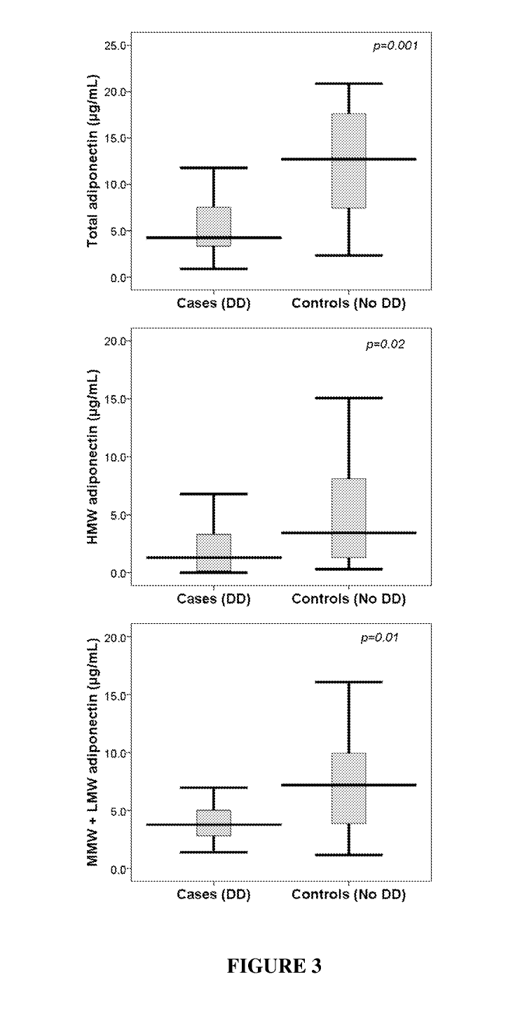

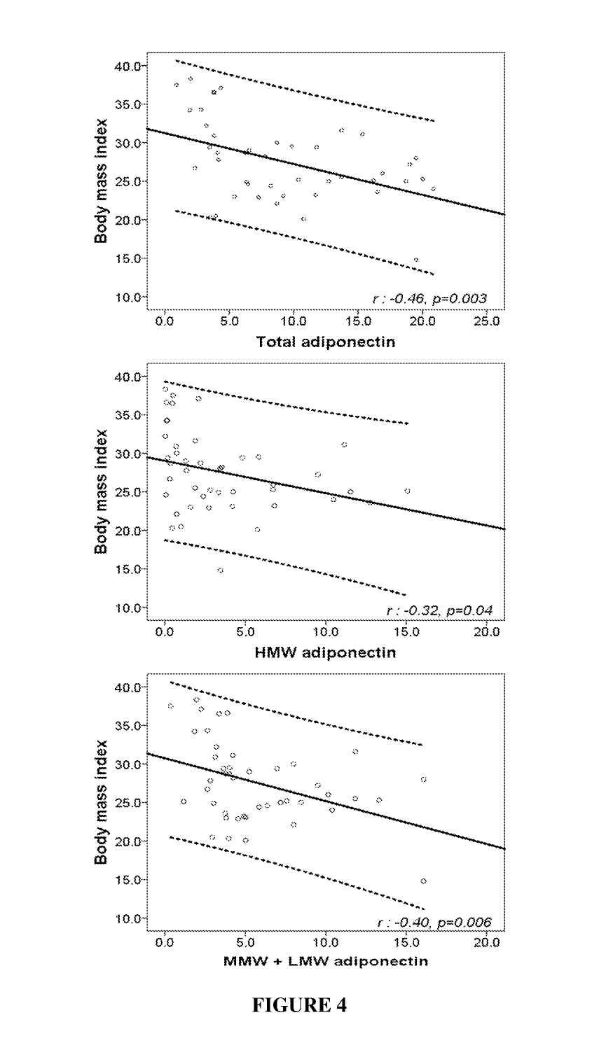

FIG. 3 compares total, HMW and MMW+LMW adiponectin levels among the cases and controls. Patients with DD had a significantly lower total adiponectin (median (IR), 4.4 (3.4-8.0) vs. 12.7 (6.2-18.7) μg/mL, p=0.001), lower HMW fraction of adiponectin (median (IR), 1.3 (0.04-3.4) vs. 3.4 (1.0-9.5) μg/mL, p=0.02), and lower MMW+LMW fraction of adiponectin (median (IR), 3.8 (2.7-5.1) vs. 7.2 (3.8-10.4) μg/mL, p=0.01). There was a moderately negative correlation of BMI with total (r: −0.46, p=0.003), HMW (r: −0.32, p=0.038) and MMW+LMW (r: −0.40, p=0.006) adiponectin levels in the study sample (FIG. 4).

Patients with DD had an independent association with both BMI (p=0.03) and total adiponectin (p<0.001) in linear regression analysis (Model 2A) using age, gender, BMI, and total adiponectin as covariates (Table 4).

TABLE 4
Linear regression model (Model 2) evaluating independent
association of adiponectin with diastolic dysfunction
using age, gender and body mass index as covariates.

| Model | Method | FR\grave{ } | t-score | |
|---|---|---|---|---|
| | Enter | 0.50 | | |
| Age | | | 0.64 | 0.35 |
| Gender | | | 1.01 | 0.10 |
| BMI | | | 2.26 | 0.03 |
| Total adiponectin | | | −4.46 | <0.001 |
| 2B | Enter | 0.39 | | |
| Age | | | 0.70 | 0.49 |
| Gender | | | 1.36 | 0.08 |
| BMI | | | 2.79 | 0.008 |
| HMW adiponectin | | | −2.99 | 0.005 |
| 2C | Enter | 0.46 | | |
| Age | | | 0.58 | 0.57 |
| Gender | | | 1.22 | 0.09 |
| BMI | | | 2.10 | 0.05 |
| MMW + LMW adiponectin | | | −3.31 | 0.002 |

BMI, Body mass index;
HMW, High molecular weight fraction;
MMW + LMW, Mid and low molecular weight fraction

Also, DD was independently associated with both BMI (p=0.008) and HMW fraction of adiponectin (p=0.005), and both BMI (p=0.05) and MMW+LMW fraction of adiponectin (p=0.002) in similar linear regression analyses (Models 2B and 2C). In all models, DD had a stronger association with adiponectin or its fractions compared to its association with BMI.

This study shows an association of low plasma adiponectin with DD. This association is independent of the age, BMI, and the existence of diabetes or hypertension. To the best of our knowledge, this is the first study that has shown this relationship in humans.

In our study, we observed an association of obesity and DD. Adiponectin was associated with DD even when including BMI in the statistical models, suggesting that adiponectin may be regulated by other factors aside from obesity.

Systolic HF and HF with preserved EF are different pathological entities that may clinically present in a similar fashion,²⁹ but their outcome with conventional heart failure therapies are different.³⁰ It is known that chronic systolic HF leads to upregulation of plasma adiponectin levels,³¹ and a higher BMI is protective in this population. Thus, finding of a low adiponectin level is useful to differentiate the two types of heart failure when echocardiography is unavailable.

In conclusion, reduced levels of adiponectin are associated with DD. Adiponectin levels may differentiate diastolic from systolic heart failure. These data suggest that raising adiponectin levels is a way to treat DD.

### Example 4

The following represents the design of a study which aims to compare markers of oxidative stress in patients with acute decompensated and chronic compensated heart failure with preserved ejection fraction (HFpEF) and heart failure with reduced ejection fraction (HFrEF). It is hypothesize that oxidative stress will be highest in acute patients and in HFrEF patients as compared to stable patients and HFpEF patients.

HFpEF accounts for approximately 30-50% of all acute heart failure admissions in North America and its prevalence is increasing.^{1,2} Furthermore, all cause mortality, readmission rates for heart failure and in hospital complication rates including cardiac arrest and acute coronary syndrome are similar between patients with HFpEF and HFrEF.² While mortality is improving for HFrEF with current therapies there are no proven therapies for HFpEF and these patients are less likely to be managed by a cardiologist.² Despite multiple studies revealing a positive correlation between HFpEF and advanced age, female sex, obesity, atrial fibrillation and hypertension and a lack of clear correlation with coronary artery disease, renal insufficiency and diabetes mellitus, the pathophysiology of diastolic dysfunction (DD), which ultimately leads to HFpEF, is poorly understood.¹⁻³

Activation of the renin-angiotensin system (RAS) is implicated in the development and progression of DD through angiotensin II (AngII) mediated inflammation, myocardial fibrosis and oxidative stress.⁴⁻⁶ Specifically, RAS activity increases production of inflammatory mediators including tumor necrosis factor alpha (TNF-α), interleukin 6 (IL-6) and transforming growth factor beta (TGF-β) leading to the stimulation of cardiac fibroblasts to produce and deposit collagen in the cardiac extracellular matrix (ECM) causing ventricular stiffness and DD.⁷⁻¹⁵ RAS activity alters the expression of matrix metalloproteinases (MMPs), tissue inhibitors of metalloproteinases (TIMPs) and collagenase further contributing to ECM fibrosis.¹⁶ Independent of fibrosis, RAS activation increases the production of reactive oxygen species (ROS) through the activation of nicotinamide adenine dinucleotide phosphate (NADPH) oxidase and uncoupling of nitric oxide synthase (NOS) resulting in decreased levels of nitric oxide (NO), which is required for diastolic relaxation.¹⁷⁻²° In animal models, inhibition of RAS reduces inflammation, fibrosis and DD through suppression of cytokine and NADPH oxidase activity.²¹⁻²⁴ In humans, increased levels of inflammatory markers are independently associated with DD.²⁵

As shown herein, early DD is not associated with RAS activation or significant oxidative stress in HFpEF excluding patients who had a history of New York Heart Association (NYHA) Class III and IV symptoms. The purpose of the study of this example is to compare clinically moderate to severe HFpEF with HFrEF in both acute and chronic patients by measuring oxidative stress markers to ultimately differentiate all four groups.

Several validated methods are available to measure oxidative stress in humans. As in our previous study, we will measure isoprostane and derivatives of reactive oxygen metabolites (D-ROMs). A weakness of our previous study could have been the use of serum measurements of isoprostane instead of urinary isoprostane which more accurately reflects overall systemic oxidative stress.³⁵ We will measure urinary excretion of 8-iso-prostaglandin F2 alpha (IPGF2), which is a chemically stable and quantitative measure of oxidative stress.³⁶ IPGF2 is a free prostaglandin isomer synthesized in vivo through free radical catalyzed peroxidation of arachidonic acid in cell membranes independent of the action of cyclooxygenase.³⁷ IPGF2 is known to correlate with the severity of HFrEF as measured by NYHA Class.^{38,39} D-ROMs are a colorimetric assay for lipid peroxidation and correlate with the presence and severity of coronary artery disease and with high sensitivity C-reactive protein (hsCRP).^{40, 41} We will also measure novel markers of oxidative stress not included in our prior study.⁴² Bilirubin is an important scavenger of ROS and biopyrrins are oxidatively modified metabolites of bilirubin.⁴³ Urinary biopyrrins correlate with the severity of HFrEF as measured by NYHA Class and pulmonary artery wedge pressure.⁴⁴ Lastly, we will measure urinary 8-hydroxy-2′-deoxyguanosine (8-OHdG), a marker of systemic oxidatively generated damage to DNA.^{45, 46 8}-OHdG correlates with the severity of ischemic HFrEF and the number of diseased vessels visualized on coronary angiography.⁴⁷ We will also measure markers of inflammation, RAS activation and lipid metabolism and record N-terminal element of brain natriuretic peptide (NT-proBNP). Biomarkers to measure:

| Oxidative Stress | Inflammation | RAS Activation | Lipid Metabolism |
|---|---|---|---|
| IPGF2 | TNF-□ | Renin | Adiponectin |
| D-ROMs | IL-6 | Aldosterone | Leptin |
| Biopyrrins | hsCRP | | |
| 8-OHdG | | | |

This study will be an observational cross-sectional cohort study, however, chronic patients will be compared to an age and sex matched control group with normal heart function as well to establish baseline levels of markers. We will enroll and consent chronic patients scheduled for outpatient appointments in the Internal Medicine and Cardiology clinics of the University of Illinois Medical Center at Chicago, Ill. (UICMC) and the Jesse Brown VA Medical Center (JB VAMC). We will enroll and consent acute patients presenting with heart failure in the Emergency Departments of UICMC and JB VAMC as well as patients admitted as inpatients within 24 hours of initial presentation. The study will involve a review of patient history, physical exam, active medications, laboratory data, electrocardiogram, echocardiography and coronary angiography as available prior to enrollment. At the time of enrollment, one blood sample drawn through peripheral venipuncture and one urine sample will be collected.

The following Inclusion Criteria will be adhered to in order to determine which patients will be included in the study:
1. Age greater than or equal to 18 years
2. Transthoracic echocardiogram within one year prior to enrollment containing tissue Doppler, mitral inflow velocities, left ventricular ejection fraction and left ventricular end-diastolic volume index data
3. Patient at UICMC or JB VAMC
4. Able to provide informed consent
5. History of admission for heart failure, need for loop diuretics and/or NYHA Class III or IV (Chronic HFpEF group only)

The following Exclusion Criteria will be adhered to in order to determine which patients will be excluded in the study:
1. Moderate to Severe Aortic or Mitral Valve Disease
2. Hemodynamically Significant Left Ventricular Outflow Tract Obstruction
3. Prosthetic Valve
4. Acute Coronary Syndrome (ACS) or ACS within 6 weeks
5. Rhythm other than Normal Sinus at Enrollment
6. Mandatory and Biventricular Pacing
7. Cardiogenic Shock
8. Active Use of Intravenous Vasodilators, Vasopressors or Inotropes
9. History of Heart Transplant or Left Ventricular Assist Device
10. Uncontrolled Hypertension (Blood Pressure>180/100 at rest) on Medications
11. Pulmonary Arterial Hypertension (Group 1)⁴⁸
12. Hemodialysis, Peritoneal Dialysis or Creatinine>2.0 mg/dL
13. Cirrhosis
14. Active Infection including Bacteremia
15. Major Trauma or Surgery within 6 weeks
16. Malignant Neoplastic Disease
17. Collagen Vascular Disease
18. Illicit drug use or alcohol abuse within 6 weeks
19. Concomitant use of investigational drug within 6 weeks
20. Systemic steroid use within 6 weeks
21. Severe disease limiting life expectancy to approximately less than 1 year

The following Procedures will be carried out in this study:

Chronic patients will be identified prior to scheduled appointments and acute patients will be identified in the Emergency Department itself or from hospital admission logs. HFpEF subjects will be identified by echocardiographic criteria if available within the year prior to enrollment and inclusive of relevant parameters to assess diastolic dysfunction as defined by the European Society of Cardiology.49 Additionally, the chronic HFpEF group's medical history will be reviewed for the presence of at least one of the following to be enrolled: previous admission to UICMC, JB VAMC or other inpatient facility for acute heart failure, history of NYHA Class III or IV functional status or the need for loop diuretics specifically for heart failure at any time. HFrEF will be identified by echocardiographic evidence of depressed left ventricular systolic function. Age and sex matched control subjects will be identified in clinic if unremarkable echocardiography is available within the year prior to enrollment.

At the time of enrollment, the subject will be educated about the study and signed informed consent will be obtained. Oxidative stress and inflammatory markers as well as other labs described previously will be obtained immediately after consent. Approximately 30 mL of blood will be obtained through peripheral venipuncture. A urine sample will also be collected. Active study participation will end at this point.

The primary outcome of the study will be comparison of oxidative stress and other biomarkers between the four identified groups and a control group. Values of these markers will also be related to clinical variables including but not limited to age, gender, smoking status, NYHA Class and specific echocardiographic measures.

There are approximately 1500 outpatient visits annually to UICMC for heart failure and approximately 400 total heart failure patients are followed as outpatients and inpatients. With the addition of recruitment of patients from JB VAMC, it will take approximately 6 months to recruit the required sample size.

Statistical Methods

This is an observational cohort study with one control group for comparison with the chronic subjects which will be age and sex matched. Factors known to affect oxidative stress such as smoking status and diabetes mellitus will be recorded and included in comparison analysis. The null hypothesis is that there is no difference in measures of oxidative stress between the four heart failure groups and in comparison with the control group as well. We will compare baseline clinical characteristics and levels of measured biomarkers between the heart failure groups and control group using student's t-test and chi-squared test for continuous and categorical variables respectively. Values between acute and chronic groups for HFpEF and HFrEF will be compared using a paired t-test. Regression analysis will be performed to determine the relationship between baseline clinical characteristics and measured variables. A p-value of <0.05 will be considered statistically significant. All analyses will be performed using SAS.

A power analysis, assuming a 10 percent loss rate due to difficulty in obtaining and processing samples, indicates that with a two-tailed alpha level of 0.05 and a test power of 0.80, the sample size required based on previous studies comparing biomarkers in acute heart failure will be 12 patients with acute HFpEF and 12 patients with acute HFrEF.50 Based on previous studies comparing biomarkers in chronic heart failure, the required sample size will be 95 patients with chronic HFpEF, 95 patients with chronic HFrEF and 14 control patients.51

Safety Monitoring and Assessment

As this is an observational study with no follow up of subjects and there is limited risk with peripheral venipuncture and urine collection, no Data Safety Monitoring Board will be required. The risks of peripheral venipuncture include bleeding, bruising, discomfort and infection at the needle insertion site. The risk of infection will be minimized by topical cleansing of the skin with an alcohol swab prior to insertion. The risk of bleeding and bruising will be limited by maintaining adequate pressure on the site after phlebotomy until hemostasis is achieved and a band aid will protect the site.

Recruitment and Consent

The principal investigator's staff will recruit subjects and collect demographic data and biological samples. Permission will be obtained from the patient's physician prior to approaching the patient in person to explain the study and obtain signed informed consent. Patients may decline to participate or withdraw at any time with no change in clinical care and with assurance of strict confidentiality. Consent will not be required prior to eligibility screening.

Biological Sample Collection

The purpose of collecting blood and urine is to measure markers of oxidative stress and inflammation which can then be correlated with clinical and echocardiographic data. A master code list will link study ID and subject identifiers. This will kept in a computer file in a password protected computer in a locked Cardiology office. Approximately 30 mL of blood and 100 mL of urine will be collected by specified study personnel directly from subjects at the time of enrollment. Samples will not be released to anyone not listed as an investigator on the protocol. Once collected, the samples will be brought to the principal investigator's laboratory in the Clinical Sciences Building of UICMC for processing within two hours. Samples will be stored in a secured −80 degree freezer until enough samples are available for analysis. Samples may be stored for up to 10 years at which point they will be destroyed by incineration. Samples will not be stored or processed at any other facility. Subjects will not be re-consented for future use of these samples. Samples will be discarded if a patient withdraws authorization at any time during the study. The data from the sample will also be deleted. Subjects will not have the option of keeping any portion of remaining samples. Study results will not be recorded in a subject's medical record at any point.

### REFERENCES

The following represents a reference list numbered according to the citation numbering used in Example 2:
(1) Owan et al., N Eng J Med 2006; 355:251-9.
(2) Zile et al., Circulation 2002; 105:1387-93.
(3) Takimoto et al., J Clin Invest 2005;115:1221-31.
(4) Cave et al., Antioxid Redox Signal 2006; 8: 691-728.
(5) Massie et al., N Eng J Med 2008; 359(23):2456-67.
(6) Yusuf et al., Lancet 2003; 362(9386):777-81.
(7) Cleland et al., Eur J Heart Fail 1999; 1(3):211-7.
(8) Jones et al., Clin Chem Acta 1998; 275:175-84.
(9) Cornelli et al., J. Nutr 131; 3208-11.
(10) Roberts et al., J Lipid Res. 2009; 50:S219-23.
(11) Neuman et al., Clin Chem 2007 ; 9:1652-7.
(12) Paulus et al., Eur Heart J 2007; 28:2539-50.
(13) Fyhrquist et al., J Hypertens Suppl 1983; Oct: 25-30.
(14) Danilov et al., J Hypertension 1996; 14; 719-27.
(15) Balyasnikova et al., J. Proteome Res 2007; 6: 1580-94.
(16) Jeney et al., Circ. Res 2005; 96; 723-9.
(17) Fukai et al., Hypertension 2009; 54; 224-5.
(18) The SOLVD Investigators. Effect of enalapril on survival in patients with reduced left ventricular ejection fractions and congestive heart failure. N Engl J Med 1991; 325:293-302.
(19) GISSI-HF investigators. Effects of Rosuvastatin in patients with chronic heart failure (GISSI-HF trial): a randomized double-blind placebo controlled trial. Lancet 2008; 372:1231-9.
(20) Lonn et al., JAMA 2005; 293:1338-47.
(21) Landmesser et al., Circulation 2000;101; 2264-2270.
(22) Hornig et al., Circulation 2001; 103:799-805
(23) Madamanchi et al., Arterioscler Thromb Vasc Biol 2005; 25:950-6.
(24) Inoue et al., Free Radic Res 2009; 43:37-46.
(25) Movahed et al., Exp Clin Cardiol 2008; 13: 141-3
(26) Tao et al., Circulation 2007; 115; 1408-16.
(27) Ouchi et al., J Biol Chem 2004; 279: 1304-9.
(28) Iacobellis et al., Am J Cardiol 2004; 15:1084-7
(29) Baker et al., Cardiovasc Diabetol 2006;5:2840-5.
(30) Adams et al., J Pharmacol Exp Ther 1983;227:749-54.

The following represents a reference list numbered according to the citation numbering used in Example 3:
1) Owan et al., N Eng J Med 2006; 355:251-59.
2) Zile et al., Circulation 2002; 105:1387-93.
3) Bhatia et al., N Engl J Med 2006; 355:260-269.
4) Massie et al., N Eng J Med 2008; 359:2456-67.
5) Yusuf et al., Lancet 2003; 362:777-81.
6) Westermann et al., Circulation 2008; 117:2051-2060.
7) Movahed et al., Exp Clin Cardiol 2008; 13: 141-43
8) Grossman et al., J Clin Invest 1975; 56:56-64.
9) Liu et al., J Am Coll Cardiol 2001; 37:1943-49.
10) Takimoto et al., et al., J Clin Invest 2005;115:1221-31.
11) Kadowaki et al., Endocr Rev 2005; 26: 439-51.
12) Pajvani et al., J Biol Chem 2003; 278:9073-85.
13) Arita et al., Biochem Biophys Res Commun 1999; 257:79-83.
14) Hotta et al., Arterioscler Thromb Vasc Biol 2000 ;20:1595-99.
15) Pischon et al., JAMA 2004 ; 291:1730-37.
16) Shimano et al., J Mol Cell Cardiol. 2010; 49: 210-20.
17) Deng et al., Int J Obes 2010: 34:165-71.
18) Fujita et al., Arterioscler Thromb Vasc Biol 2008; 28:863-70.
19) Fujioka et al., Am J Physiol Heart Circ Physiol 2006 ; 290 :240-16.
20) Shibata et al., Nat Med 2004; 10:1384-89.
21) Paulus et al., Eur Heart J 2007; 28:2539-50.
22) Sam et al., Endocrinology 2010; 151:322-31.
23) Silberman et al., Circulation 2010; 121:519-28.
24) Tao et al., Circulation 2007; 115:1408-16.
25) Yatagai et al., Metabolism 2003; 52:1274-8.
26) Iacobellis et al., Am J Cardiol 2004; 15:1084-7
27) Aydin et al., Metab Syndr Relat Disord 2010;8:229-234.
28) Karastergiou et al., Arterioscler Thromb Vasc Biol 2010; 30:1340-6.
29) Ezekowitz et al., Am J Cardiol 2008; 102:79-83.
30) Zile et al., Circulation 2010; 121:1393-405.
31) Kistorp et al., Circulation 2005; 112:1756-62.
32) Imbeault et al., Clin Endocrinol 2004; 60:429-33.

The following represents a reference list numbered according to the citation numbering used in Example 4:
1. Owan et al., N Engl J Med 2006;355(3):251-259.
2. Bhatia et al., N Engl J Med 2006;355(3):260-269.
3. Zile et al., N Engl J Med 2004;350(19):1953-1959.
4. Kai et al., Hypertens Res 2005;28(6):483-490.
5. Martos et al., Circulation 2007;115(7):888-895.
6. Cave et al., Antioxid Redox Signal 2006;8(5-6):691-728.
7. Brilla et al., Am J Cardiol 1995;76(13):8D-13D.
8. Brilla et al., Clin Investig 1993;71(5 Suppl):S35-S41.
9. Weber et al., Hypertension 2004;43(4):716-719.
10. Tokuda et al., Hypertension 2004;43(2):499-503.
11. Phillips et al., Curr Opin Investig Drugs 2002;3(4):569-577.
12. Nicoletti et al., Cardiovasc Res 1996;32(6):1096-1107.
13. Lee et al., J Mol Cell Cardiol 1995;27(10):2347-2357.
14. Gray et al., Cardiovasc Res 1998;40(2):352-363.
15. Campbell et al., J Mol Cell Cardiol 1997;29(7):1947-1958.
16. Deschamps et al., Cardiovasc Res 2006;69(3):666-676.
17. Takimoto et al., J Clin Invest 2005;115(5):1221-1231.
18. Mehta et al., Am J Physiol Cell Physiol 2007;292(1):C82-C97.
19. Ruf et al., Pflugers Arch 2002;443(3):483-490.
20. Silberman et al., Circulation 2010;121(4):519-528.
21. Wu et al., Circulation 2001;104(22):2716-2721.
22. Oudit et al., Cardiovasc Res 2007;75(1):29-39.
23. Tokuda et al., J Cardiovasc Pharmacol 2003;42 Suppl 1:S61-S65.
24. Nishio et al., J Hypertens 2007;25(2):455-461.
25. Sciarretta et al., Am J Hypertens 2007;20(7):784-791.
26. Yusuf et al., Lancet 2003;362(9386):777-781.
27. Massie et al., N Engl J Med 2008;359(23):2456-2467.
28. Cleland et al., Eur Heart J 2006;27(19):2338-2345.
29. Shah et al., J Card Fail 2010;16(3):260-267.
30. The SOLVD Investigators. N Engl J Med 1991;325(5):293-302.
31. Flather et al., Lancet 2000;355(9215):1575-1581.
32. Devereux et al., Circulation 2004;110(11):1456-1462.
33. Solomon et al., Lancet 2007;369(9579):2079-2087.
34. Smita Negi MD, Irfan Shukrullah MBBS, Emir Veledar PhD et al. Renin-Angiotensin Activation and Oxidative Stress in Early Diastolic Dysfunction. Submitted. 2010. Ref Type: Unpublished Work
35. Nourooz-Zadeh, Biochem Soc Trans 2008;36(Pt 5):1060-1065.
36. Milne et al., Methods Enzymol 2007;433:113-126.
37. Nonaka-Sarukawa et al., Heart 2003;89(8):871-874.
38. Polidori et al., J Card Fail 2004;10(4):334-338.
39. Radovanovic et al., Redox Rep 2008;13(3):109-116.
40. Cornelli et al., J Nutr 2001;131(12):3208-3211.
41. Kamezaki et al., J Atheroscler Thromb 2008;15(4):206-212.
42. Braunwald, N Engl J Med 2008;358(20):2148-2159.
43. Stocker, Free Radic Res Commun 1990;9(2):101-112.
44. Hokamaki et al., J Am Coll Cardiol 2004;43(10):1880-1885.
45. Kasai et al., Nucleic Acids Res 1984;12(4):2137-2145.
46. Wu et al., Clin Chim Acta 2004;339(1-2):1-9.
47. Nagayoshi et al., Free Radic Res 2009;43(12):1159-1166.
48. Simonneau et al., J Am Coll Cardiol 2009;54(1 Suppl):S43-S54.
49. Paulus et al., Eur Heart J 2007;28(20):2539-2550.
50. Dieplinger et al., Heart 2009;95(18):1508-1513.
51. Stahrenberg et al., Eur J Heart Fail 2010.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed:

1. A method of diagnosing diastolic dysfunction in the absence of systolic dysfunction in a subject exhibiting a sign or symptom of heart failure, comprising assaying a sample obtained from the subject for
a. evidence of activation of renin-angiontensin system (RAS),
b. evidence of oxidative stress,
c. a level of adiponectin, or
d. a combination thereof,
wherein, when there is a lack of evidence of RAS activation, a lack of evidence of oxidative stress, a reduction in the level of adiponectin, or a combination thereof, as compared to a control subject exhibiting a sign or symptom of heart failure, the subject is diagnosed with diastolic dysfunction in the absence of systolic dysfunction.

2. A method of diagnosing a type of heart failure in a subject suffering from a heart failure, comprising assaying a sample obtained from the subject for
a. evidence of activation of renin-angiontensin system (RAS),
b. evidence of oxidative stress,
c. a level of adiponectin, or
d. a combination thereof,
wherein, when there is a lack of evidence of RAS activation, a lack of evidence of oxidative stress, a reduction in the level of adiponectin, or a combination thereof, as compared to a control subject suffering from heart failure, the subject is diagnosed with heart failure with preserved ejection fraction.

3. A method of determining a therapeutic regimen for a subject exhibiting a sign or symptom of heart failure, comprising assaying a sample obtained from the subject for
a. evidence of activation of renin-angiontensin system (RAS),
b. evidence of oxidative stress,
c. a level of adiponectin, or
d. a combination thereof,
wherein, when there is a lack of evidence of RAS activation, a lack of evidence of oxidative stress, a reduction in the level of adiponectin, or a combination thereof, as compared to a control subject exhibiting a sign or symptom of heart failure, the therapeutic regimen is determined to be a therapeutic regimen for treating diastolic dysfunction in the absence of systolic dysfunction.

4. A method of treating a subject for diastolic dysfunction in the absence of systolic dysfunction, comprising
a. assaying a sample obtained from the subject for
i. evidence of activation of renin-angiontensin system (RAS),
ii. evidence of oxidative stress,
iii. a level of adiponectin, or
iv. a combination thereof, and
b. administering to the subject a therapeutic agent suitable for treating diastolic dysfunction in the absence of systolic dysfunction in an amount effective to treat the diastolic dysfunction.

5. The method of any of the preceding claims, wherein assaying the sample for evidence of RAS activation comprises assaying for one or more positive RAS markers, one or more negative RAS markers, or a combination thereof.

6. The method of claim 5, wherein assaying for one or more positive RAS markers comprises assaying the sample for an amount or activity level of renin, angiotensin II, aldosterone, angiotensin-converting enzyme (ACE), NADPH oxidase, or a combination thereof.

7. The method of claim 6, comprising assaying for ACE amounts or ACE activity levels.

8. The method of any of the preceding claims, wherein assaying the sample for evidence of oxidative stress comprises assaying for one or more positive oxidative stress markers, one or more negative oxidative stress markers, or a combination thereof.

9. The method of claim 8, wherein assaying for one or more positive oxidative stress markers comprises assaying for an amount or activity level of a reactive oxygen species, glutathione disulfide (GSSG), oxidized cystine (CysS), a lipid peroxidase, an isoprostane, nitrite, nitrate, plasminogen activator inhibitor 1 (PAI-1), dihydrobiopterin (BH₂), uncoupled nitric oxide synthse (uncoupled NOS), or a combination thereof.

10. The method of claim 9, wherein assaying for one or more negative oxidative stress markers comprises assaying for an amount or activity level of glutathione (GSH), cysteine (Cys), nitric oxide (NO), a coupled nitric oxide synthase (coupled NOS), tetrahydrobiopterin (BH₄), or a combination thereof.

11. The method of any of claims 8 to 10, comprising assaying for levels of GSH, GSSG, Cys, CysS, DROM, isoprostane, or a combination thereof.

12. The method of any of the preceding claims, comprising assaying the sample for angiotensin I, angiotensinogen, anti-diuretic hormone (ADH), leptin, resistin or a combination thereof.

13. The method of any of the preceding claims, wherein assaying for a level of adiponectin comprises assaying the sample for a total level of adiponectin, a level of high molecular weight (HMW) adiponectin, a level of mid molecular weight (MMW) adiponectin, a level of low molecular weight (LMW) adiponectin, or a combination thereof.

14. The method of claim 13, comprising assaying the sample for a total level of adiponectin and a level of HMW adiponectin.

15. The method of any of the preceding claims, wherein the sample is a blood sample, plasma sample, serum sample, urine sample, or a composite panel sample comprising at least two of a blood sample, a plasma sample, a serum sample, and a urine sample.

16. The method of any of the preceding claims, comprising assaying for a combination of evidence for RAS activation and evidence of oxidative stress.

17. The method of any of the preceding claims, further comprising performing echocardiography, tissue Doppler imaging, cardiac catheterization, or magnetic resonance imaging, or a combination thereof.

18. The method of any of claims 1 and 3 to 17, wherein the diastolic dysfunction in the absence of systolic dysfunction is an early diastolic dysfunction in the absence of diastolic dysfunction.

19. The method of any of the preceding claims, wherein the subject is a mammal.

20. The method of claim 19, wherein the mammal is a human.

21. The method of claim 20, wherein the human is a male human.

22. The method of claim 20 or 21, wherein the human has a BMI of about 29 or higher.

23. The method of any of claims 1 and 3 to 22, wherein the subject is suffering from heart failure.

24. The method of any of claims 1 to 3 and 5 to 23, wherein the heart failure is an NYHA Class I or Class II heart failure.

25. The method of any of the preceding claims, wherein the subject suffers from obesity, hypertension, diabetes, or a combination thereof.

26. The method of any of claims 1 to 3 and 5 to 25, wherein the control subject does not suffer from diastolic dysfunction.

27. The method of any of claims 1 to 3 and 5 to 26, wherein the control subject suffers from systolic dysfunction.

28. The method of any of the preceding claims, wherein the sign or symptom of heart failure is dyspnea, peripheral edema, pulmonary vascular redistribution, interstitial edema, pleural effusions, or a combination thereof.

29. The method of any of claims 4 to 28, wherein the therapeutic agent increases the level of adiponectin in the subject.

30. A kit comprising instructions for diagnosing diastolic dysfunction in the absence of systolic dysfunction and one or more of:
a. a binding agent or substrate specific for a positive RAS marker;
b. a binding agent or substrate specific for a negative RAS marker;
c. a binding agent or a substrate specific for a positive oxidative stress marker;
d. a binding agent or a substrate specific for a negative oxidative stress marker; and
e. a binding agent specific for adiponectin.

31. A method of treating diastolic dysfunction in the absence of systolic dysfunction in a subject, comprising administering to the subject an agent which increases the level of adiponectin in the subject.

32. A method of treating or preventing heart failure with preserved ejection fraction in a subject, comprising administering to the subject an agent which increases the level of adiponectin in the subject.

33. The method of claims 31 and 32, wherein the agent is selected from the group consisting of an adiponectin protein, a functional equivalent thereof, a nucleic acid molecule encoding an adiponectin protein or functional equivalent thereof.


SEQUENCE LISTING (1 sequence)

SEQ ID NO: 1 - protein, 230 aa, Homo sapiens
GHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN